(19) United States
(12) Patent Application Publication
(10) Pub. No.: US 2023/0233489 A1
(43) Pub. Date: Jul. 27, 2023

(54) TREATMENT OF VIRAL INFECTIONS WITH ESTROGEN RECEPTOR MODULATORS AND ANTI-INFLAMMATORY AGENTS

(71) Applicant: THE CLEVELAND CLINIC FOUNDATION, Cleveland, OH (US)

(72) Inventors: Feixiong Cheng, Cleveland, OH (US); Reena Mehra, Cleveland, OH (US); Sujata Rao, Cleveland, OH (US); Yadi Zhou, Cleveland, OH (US); Yuan Hou, Cleveland, OH (US)

(21) Appl. No.: 18/001,832

(22) PCT Filed: Jun. 18, 2021
(86) PCT No.: PCT/US2021/037999

§ 371 Date: Dec. 14, 2022

Related U.S. Application Data
(60) Provisional application No. 63/040,620, filed on Jun. 18, 2020.

(51) Int. Cl. A61K 31/138; A61K 31/4045; A61P 31/14; A61K 9/00

(52) U.S. Cl. CPC A61K 31/138; A61K 31/4045; A61P 31/14; A61K 9/0053

(57) ABSTRACT

The present invention provides methods, compositions, combinations, and kits for treating a subject with a viral infection (e.g., COVID-19) by administering or providing an estrogen receptor modulator and an anti-inflammatory agent or by administering or providing an anti-inflammatory agent (e.g., melatonin) to the subject.

### CROSS-REFERENCE TO RELATED APPLICATIONS

This application claims the benefit of U.S. Provisional Application No. 63/040,620, filed Jun. 18, 2020, the content of which is herein incorporated by reference in its entirety.

### STATEMENT REGARDING FEDERAL FUNDING

This invention was made with government support under HL138272 and AG066707 awarded by the National Institutes of Health. The government has certain rights in the invention.

### FIELD

The present invention provides methods, compositions, combinations, and kits for treating a subject with a viral infection (e.g., COVID-19) by administering or providing an estrogen receptor modulator and an anti-inflammatory agent to the subject or administering or providing an anti-inflammatory agent (e.g. melatonin) to the subject.

### BACKGROUND

Coronavirus disease 2019 (COVID-19) is an infectious disease caused by severe acute respiratory syndrome coronavirus 2 (SARS-CoV-2). It was first identified in December 2019 in Wuhan, China, and has since spread globally, resulting in an ongoing pandemic. Common symptoms include fever, cough, fatigue, shortness of breath, and loss of smell and taste. While the majority of cases result in mild symptoms, some progress to an unusual form of acute respiratory distress syndrome (ARDS) likely precipitated by cytokine storm, multi-organ failure, septic shock, vascular inflammation, and blood clots. The time from exposure to onset of symptoms is typically around five days but may range from two to fourteen days. The virus is primarily spread between people during close contact, most often via small droplets produced by coughing, sneezing, and talking. People may also become infected by touching a contaminated surface and then touching their face. According to the World Health Organization, there are no available vaccines nor specific antiviral treatments for COVID-19.

### SUMMARY OF THE INVENTION

Disclosed herein are methods, compositions, combinations, and kits for treating a subject with a viral infection (e.g., coronavirus infection) by administering or providing a therapeutically effective amount of a selective estrogen receptor modulator, or a pharmaceutically acceptable salt thereof, and a therapeutically effective amount of an anti-inflammatory agent, or a pharmaceutically acceptable salt thereof The estrogen receptor modulator and anti-inflammatory agent may be administered simultaneously or sequentially in any order.

In some embodiments, the estrogen receptor modulator is selected from the group consisting of: tamoxifen, toremifene, clomifene, and pharmaceutically acceptable salts, variants, and combinations thereof. In select embodiments, the estrogen receptor modulator is toremifene, or a pharmaceutically acceptable salt thereof (e.g. toremifene citrate). In some embodiments, the anti-inflammatory agent is melatonin.

In some embodiments, the virus is selected from the group consisting of severe acute respiratory syndrome coronavirus-1 (SARS-CoV-1), severe acute respiratory syndrome coronavirus-2 (SARS-CoV-2), Middle East respiratory syndrome coronavirus (MERS-CoV), and Ebola virus. In some embodiments, the virus is a coronavirus.

In some embodiments, the subject has or is suffering from human coronavirus disease 2019 (COVID-19). In some embodiments, the subject is a human. In some embodiments, the subject has lung inflammation. In some embodiments, the subject has general body inflammation. In certain embodiments, the subject is on a ventilator.

Also disclosed herein is a combination or kit comprising a selective estrogen receptor modulator, or a pharmaceutically acceptable salt thereof and an anti-inflammatory agent, or a pharmaceutically acceptable salt thereof. In some embodiments, the estrogen receptor modulator is selected from the group consisting of: tamoxifen, toremifene, clomifene, and pharmaceutically acceptable salts, variants, or combinations thereof. In select embodiments, the estrogen receptor modulator is toremifene, or a pharmaceutically acceptable salt thereof (e.g. toremifene citrate). In some embodiments, the anti-inflammatory agent is melatonin. In some embodiments, the selective estrogen receptor modulator, or a pharmaceutically acceptable salt thereof, and/or the anti-inflammatory agent, or a pharmaceutically acceptable salt thereof, are provided in a pharmaceutical composition.

Other aspects and embodiments of the disclosure will be apparent in light of the following detailed description and accompanying figures.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a schematic of the overall workflow of a network-based methodology for drug repurposing and rational design of drug combinations for COVID-19. (A) SARS-CoV-2 host proteins were identified from literature sources and pooled to generate a pan-coronavirus (CoV) protein subnetwork. (B) Network proximity between drug targets and CoV proteins was calculated to screen for repurposable drugs for SARS-CoV-2 under the human protein interactome model. (C & D) Gene set enrichment analysis was utilized to validate the network-based prediction. (E) Top candidates were further prioritized for drug combinations using network-based method captured by the ‘Complementary Exposure’ pattern: the targets of the drugs both hit the CoV-host sub-network but target separate neighborhoods in the human interactome network. (F) Overall hypothesis of the network-based methodology.

FIG. 2 shows a schematic of the network-based rational design of effective drug combinations for COVID-19. (A) Four possible exposure modes of the COVID-19 disease module to the targets of pairwise drug combinations. An effective drug combination can be captured by the ‘Complementary Exposure’ pattern: the targets of the drugs both hit the COVID-19 disease module, but target separate neighborhoods in the human protein-protein interactome network. Z_{CA} and ZcB denote the network proximity (Z-score) between drug targets (Drugs A and B) and COVID-19 module. S_{AB} denotes separation score of drug targets between Drug_{A} and Drug_{B}. (B and C) A ‘Complementary Exposure’ pattern of melatonin plus toremifene on the COVID-19 module.

FIG. 3 shows a computational biophysical simulation demonstrating that toremifene (ball-stick) specifically blocks interaction between the spike protein of SARS-CoV-2 and human ACE2.

FIG. 4 is a schematic of a model that combines antiviral (e.g. toremifene) and anti-inflammatory (e.g. melatonin) agents for effective treatment of COVID-19. Melatonin, a synthesized hormone, originated ˜2.5 billion years ago and is evolutionarily conserved in all organisms from bacteria to humans. Toremifene, a selective estrogen receptor modulator approved by FDA for the treatment of advanced breast cancer, has shown various antiviral activities across Ebola virus, MRES-CoV, SARS-CoV-1, and SARS-CoV-2.

FIGS. 5A-5D show a schematic of the overall workflow. (A) A diagram illustrating the basic pathogenesis of SARS-CoV-2. (B) A diagram illustrating how to build a global interactome map for SARS-CoV-2. (C) A diagram illustrating network-based measure of disease manifestations associated with COVID-19. (D) A workflow illustrating validation of network-based findings.

FIGS. 6A-6I is a network and biological characteristics of the SARS-CoV-2 interactome map. (A) Pathway and gene ontology (biological process) enrichment analysis results of the SARS-CoV-2 host genes/proteins across five different data sets. (B, C, D) Network and biological characteristics of the SARS-CoV-2 host genes/proteins. The proteins in PanCoV-PPI had higher node degrees (B), lower non-synonymous to synonymous substitutions (dN/dS) ratios (C), and lower evolutionary ratios (D) compared to randomly selected proteins (grey, mean±standard deviation of 100 repeats). (E) Among the 460 proteins in PanCoV-PPI, 450 (98%) are expressed in lungs, and 317 (69%) are lung specific expression (Z>0). (F, G) The distribution of the node degrees in the human interactome and dN/dS ratios of PanCoV-PPI and four published virus-related host protein sets. (H) The shared target human proteins (blue) of SARS-CoV-2 (red) and other viruses (green). (I) SARS-CoV-2 target proteins overlap significantly with disease-associated genes (Mendelian disease and Orphan disease), cancer genes, cell cycle genes, and innate immune genes.

FIGS. 7A-7B is a global network illustrating disease manifestations associated with human coronavirus. (A) The target human proteins of SARS-CoV-2 are connected to the disease-associated proteins. Blue links (edges) indicate physical protein-protein interactions. For SARS-CoV-2, its viral proteins are shown by light red node. The target human proteins (blue) of the viruses are intricately connected to the disease-associated proteins (green). Human disease nodes are colored by different disease categories: autoimmune, cancer, cardiovascular, metabolic, neurological, and pulmonary. (B) Estimation of the pooled risk ratio using random effects meta-analysis for 10 comorbidities between severe versus non-severe COVID-19 patients. The tau² and I² statistics were calculated to quantify the heterogeneity among studies. I²<50% was considered as low heterogeneity among studies, 50% <I²<75% was considered as moderate heterogeneity, and I²>75% was considered as high heterogeneity.

FIGS. 8A-8D shows landscape of disease manifestations associated with COVID-19 quantified by network proximity measure. (A) Heatmaps showing the network proximities of COVID-19 with 64 diseases across 6 categories. The network proximities of the disease modules and the five SARS-CoV-2 data sets were evaluated using the “closest” network proximity measure (see Methods). The magnitude of the proximity is indictive of their biological relationship: closer network proximity of SARS-CoV-2 host genes/proteins of a disease indicates higher potential of manifestation between COVID-19 and a specific disease. P<0.05 computed by permutation test was considered significant (indicated by horizontal bars). Three categories, autoimmune, pulmonary, and neurological frequently show significant proximities to COVID-19. Inflammatory bowel disease, attention-deficit/hyperactivity disorder, and stroke achieved significance with all five SARS-CoV-2 data sets. (B, C) Highlighted subnetworks between SARS-CoV-2 host genes/proteins with the disease-associated proteins of respiratory distress syndrome (B) and sepsis (C). (D) Clinical data analyses showed an association of COVID-19 severity with IL-6 expression levels in patients. Meta-analysis of random effects model was performed using the mean difference in IL-6 (pg/ml). There was a high heterogeneity among these studies (I²=94%, P<0.001).

FIGS. 9A-9H shows the Endophenotypes between asthma and COVID-19. (A) A highlighted subnetwork between the asthma-associated genes, differential metabolites in asthma, and SARS-CoV-2 host genes, under the human interactome network model. (B) A heatmap highlighting differential gene expression analyses for the genes identified in asthma and COVID-19 subnetwork analysis (A). Differential gene expression analysis was performed using two existing asthma cohorts (GSE63142 and GSE130499). SvsC, severe versus control; MvsC mild versus control; SvsM, severe versus mild. Blue bars show the node degree enrichment in the subnetwork (A) compared to a random network of the same size (see Methods). Black dotted line indicates enrichment=1. (C) UMAP visualization for human bronchial epithelial cells. (D) Cell type-specific expression levels of ACE2 and TMPRSS2 across 14 cell types in human bronchial epithelial cells. (E) Cell type-specific expression levels of seven highlighted inflammatory genes (A and B) show elevated expression levels in secretory 3 cells compared to other cell types. (F) UMAP visualization for lung cells. (G) Cell type-specific expression levels of ACE2 and TMPRSS2 across 9 cell types in lung cells. (H) Cell type-specific expression levels of four highlighted inflammatory genes (A and B) show elevated expression levels in alveolar type II cells compared to other cell types.

FIGS. 10A-10I show inflammatory endophenotype between inflammatory bowel disease (IBD) and COVID-19. (A) Severe COVID-19 patients have higher risks of abdominal pain and diarrhea by meta-analysis. (B) A highlighted subnetwork between the IBD-associated genes and the SARS-CoV-2 virus proteins and virus-host (human) proteins under the human interactome network model. (C) UMAP visualization of non-epithelial cells from the ileal tissues of patients with Crohn's disease. (D) UMAP visualization of epithelial cells from the ileal tissues of patients with Crohn's disease. (E) Cell type-specific expression of ACE2 and TMPRSS2 in non-epithelial cells (C). (F) Cell type-specific expression of ACE2 and TMPRSS2 in epithelial cells (D). (G) The co-expression of ACE2 and TMPRSS2 are elevated in absorptive enterocytes of the inflamed ileal tissues compared to uninflamed tissues in patients with Crohn's disease. (H) Box plot showing the expression of ACE2 and TMPRSS2 in absorptive enterocytes expressing ACE2 and TMPRSS2, respectively. (I) Co-expression analysis for the genes in the subnetwork with ACE2 and TMPRSS2. Heatmap shows the Pearson correlation coefficients (PCC) of ACE2 and TMPRSS2 with other genes (labeled in B) in the absorptive enterocytes. Blue bars show the degree enrichment of the genes in the subnetwork compared to a random network of the same size. Black dotted line indicates enrichment=1.

FIG. 11A and 11B show network-based prediction and patient-based validation of drug repurposing for COVID-19. FIG. 11A shows thirty-four drugs from the top predicted list are highlighted with the disease category they are approved by U.S. FDA. Three types of evidences were highlighted: (i) network proximities of drug's targets across the four SARS-CoV-2 data sets (SARS2-DEG, SARS2-DEP, HCoV-PPI, and SARS2-PPI) in the human interactome; (ii) gene set enrichment analysis (GSEA) scores across five coronavirus transcriptomics and proteomics data sets (see Methods), and (iii) literature-reported antivirus profiles. GSEA scores shown in grey indicate that these drugs cannot be evaluated due to the lack of data. Five drugs that are currently being or have been tested in clinical trials are noted. Horizontal bars indicate P<0.05. ES, enrichment score by GESA analysis (see Methods). HIV, human immunodeficiency virus. EBOV, Zaire ebolavirus. RV, rhinovirus. HAV, hepatitis A virus. HBV, hepatitis B virus. HCV, hepatitis C virus. HDV, hepatitis D virus. SARS-CoV-2, severe acute respiratory syndrome coronavirus 2. SARS-CoV, severe acute respiratory syndrome coronavirus. MERS-CoV, Middle East respiratory syndrome coronavirus. HCoV-229E, human coronavirus 229E. MHV, mouse hepatitis virus. IAV, influenza A virus. IBV, influenza B virus. WNV, West Nile virus. ZIKV, Zika virus. ANDV, Andes virus. CHIKV, chikungunya virus. CMV, cytomegalovirus. DENV, dengue virus. EMCV, encephalomyocarditis virus. MV, measles virus. RSV, respiratory syncytial virus. SVCV, spring viremia of carp virus. FIG. 11B shows that melatonin is associated with a 50-60% reduced likelihood of SARS-COV-2 from the Cleveland Clinic COVID-19 registry. Propensity score-matching cohort studies were performed (1,857 positive and 18,363 COVID-19 negative patients tested by nasopharyngeal swab test) by adjusting age, sex, race, and comorbidities (hypertension, diabetes, heart disease, and various chronic pulmonary conditions). ARB: angiotensin II receptor blockers; ACEIs: angiotensin-converting enzyme inhibitors.

FIGS. 12A-12D show patient-based validation of drug repurposing for COVID-19. Validation for (A) melatonin and (B) carvedilol using the whole COVID-19 registry (all combined population). Validation for melatonin (C) in the black American (African American) subgroup and (D) in the white American subgroup. Patient groups were matched using propensity score matching. Four models were evaluated: (1) model 1 was matched using age, sex, race, and smoking without adjustment for the odds ratio; (2) model 2 was matched using age, sex, race, and smoking, and the odds ratio of COVID-19 was adjusted by age, sex, race, and smoking; (3) model 3 was matched using age, sex, race, smoking, coronary artery disease, diabetes, hypertension, and COPD without adjustment for the odds ratio; and (4) model 4 was matched using age, sex, race, smoking, coronary artery disease, diabetes, hypertension, and COPD, and the odds ratio of COVID-19 was adjusted by age, sex, race, smoking, coronary artery disease, diabetes, hypertension, and COPD. These models were adjusted for different variables using the propensity score matching approach. ACEI, angiotensin-converting enzyme inhibitor; ARB, angiotensin II receptor blocker; COPD, chronic obstructive pulmonary disease; OR, odds ratio.

### DETAILED DESCRIPTION

The present disclosure provides methods, compositions, combinations, and kits for treating a subject with a viral infection (e.g., coronavirus infection) by administering or providing at least one or both of: a therapeutically effective amount of a selective estrogen receptor modulator, or a pharmaceutically acceptable salt thereof, and a therapeutically effective amount of an anti-inflammatory agent, or a pharmaceutically acceptable salt thereof. In some embodiments, the methods comprise administering or providing a therapeutically effective amount of a selective estrogen receptor modulator, or a pharmaceutically acceptable salt thereof. In some embodiments, the methods comprise administering or providing a therapeutically effective amount of an anti-inflammatory agent, or a pharmaceutically acceptable salt thereof. In some embodiments, the methods comprise administering or providing both a therapeutically effective amount of a selective estrogen receptor modulator, or a pharmaceutically acceptable salt thereof, and a therapeutically effective amount of an anti-inflammatory agent, or a pharmaceutically acceptable salt thereof.

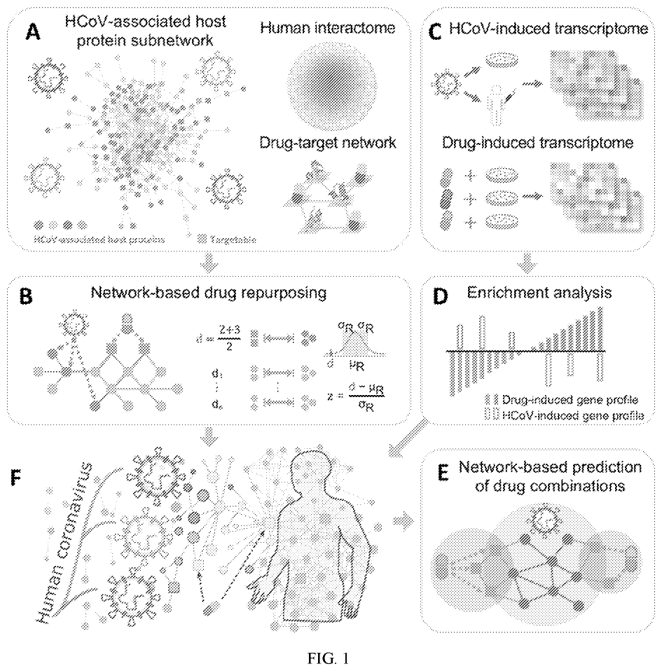

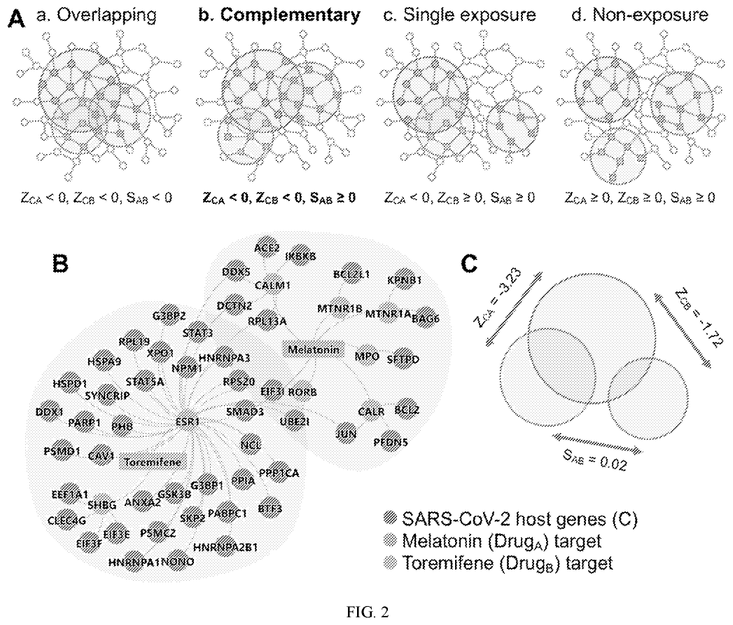

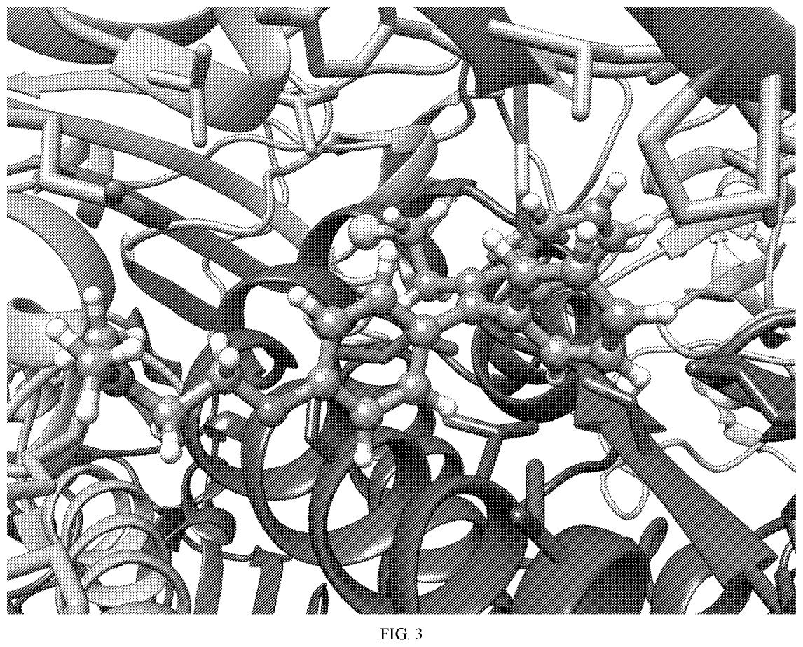

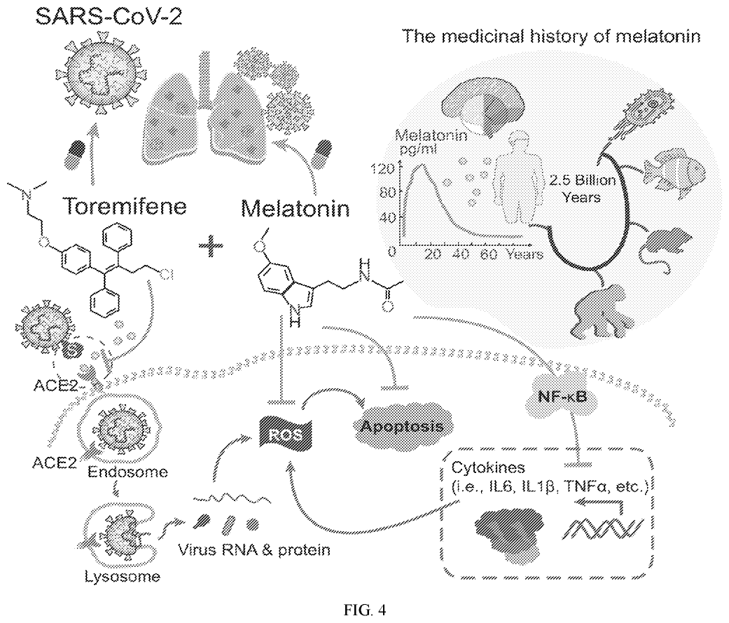

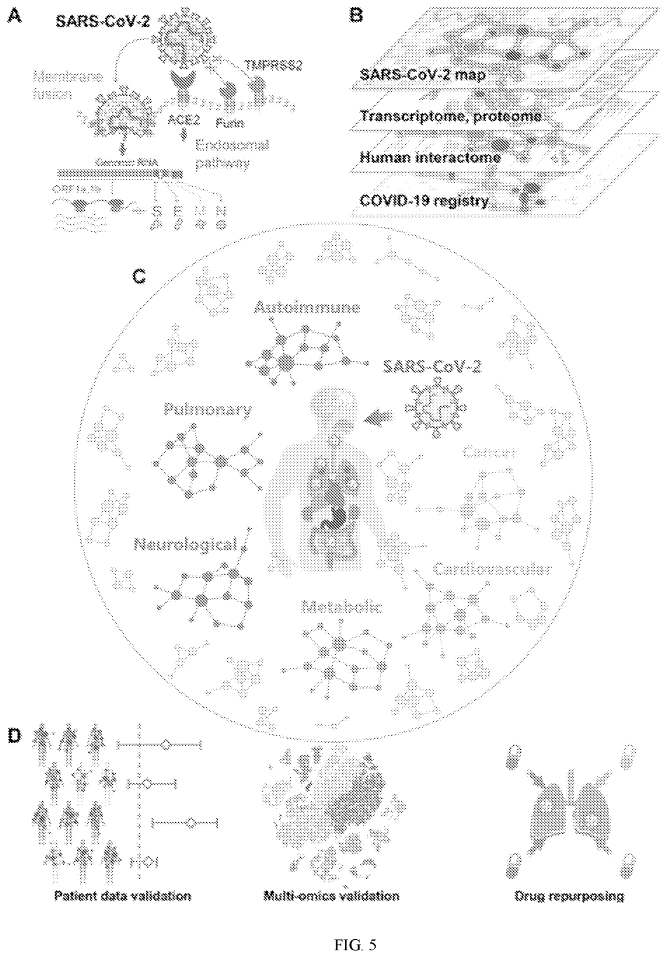

SARS-CoV-2 employs host cellular factors (e.g. angiotensin-converting enzyme 2 [ACE2]) for successful replication during infections. Systematic identification of virus-host protein-protein interactions (PPI) is an effective way toward targeting cellular virus-host interactome and offers a strategies for the development of effective treatment for COVID-19. Using network-based complementary exposure prediction, a combination therapy of melatonin and toremifene was identified as having synergistic effects in the treatment of patients positive for COVID-19 (FIG. 1).

Toremifene, a first generation nonsteroidal selective estrogen receptor modulator (SERM), has been shown to block various viral infections at low micromolar levels, including Ebola virus (50% inhibitive concentration [IC₅₀]=˜1 μM), MERS-CoV (50% effective concentration [EC₅₀]=12.9 μM), SARS-CoV-1 (EC₅₀=11.97 μM), and SARS-CoV-2 (IC₅₀=3.58 μM). Importantly, toremifene significantly improved survival rate of Ebola virus-infected mice models. Toremifene has been approved for the treatment of advanced breast cancer in post-menopausal women and has also been studied in men with prostate cancer (˜1,500 subjects) with reasonable tolerability. Toremifene is 99% bound to plasma protein with good bioavailability and typically administered at a dosage of 60 mg orally.

Melatonin regulates circadian rhythms, a homeostatic mechanism comprised of interacting cellular clocks and environmental influences rhythmically altering translation of genes responsible for melatonin-mediated anti-inflammatory and immune-related effects. Moreover, melatonin suppresses NLRP3 inflammasome activation induced by cigarette smoking 3 and attenuates pulmonary inflammation, not only via reduction of TNF-a expression, but also via increase in anti-inflammatory cytokine interleukin IL-6 and IL-10.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “treat,” “treating” and the like means a slowing, stopping or reversing of progression of a disease or disorder when provided in a composition described herein to an appropriate subject. The term also includes a reversing of the progression of such a disease or disorder to a point of eliminating or greatly reducing the disease. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

As used herein, the terms “providing”, “administering,” “introducing,” are used interchangeably herein and refer to the placement of the active agents or compositions of the disclosure into a subject by a method or route which results in at least partial localization of the composition to a desired site. The compositions can be administered by any appropriate route which results in delivery to a desired location in the subject.

As used herein, the term “subject” refers to any animal (e.g., a mammal), including, but not limited to, humans and non-human animals. The terms “subject” and “patient” may be used interchangeably herein in reference to a human subject. The term “non-human animals” refers to all non-human animals including, but are not limited to, vertebrates such as rodents, non-human primates, ovines, bovines, ruminants, lagomorphs, porcines, caprines, equines, canines, felines, ayes, etc. The subject may include either adults or juveniles (e.g., children). In some embodiments, the subject is a human.

The terms “effective amount” or “therapeutically effective amount,” as used herein, refer to a sufficient amount of an agent or a composition or combination of agents/compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the active agent required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment).

The term “pharmaceutically acceptable salt” refers to salts or zwitterions of compound which are water or oil-soluble or dispersible, suitable for treatment of disorders without undue toxicity, irritation, and allergic response, commensurate with a reasonable benefit/risk ratio and effective for their intended use. Salts may be commercially available or may be prepared during the final isolation and purification of the compounds, or separately by reacting the amino group of memantine with a suitable acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. In some embodiments, toremifene is administered as toremifene citrate.

### 2. Methods

Disclosed herein are methods of treating a viral infection in a subject. The methods comprise administering a therapeutically effective amount of a selective estrogen receptor modulator, or a pharmaceutically acceptable salt thereof, and a therapeutically effective amount of an anti-inflammatory agent, or a pharmaceutically acceptable salt thereof, to the subject.

Estrogen receptor modulators include, but are not limited to, triphenylethylene derivatives (e.g. tamoxifen, toremifene, droloxifene, 3 25 hydroxytamoxifen, idoxifene, TAT-59 (a phosphorylated derivative of 4- hydroxytamoxifen) and GW5638 (a carboxylic acid derivative of tamoxifen)); non-steroidal estrogen receptor modulators (e.g. raloxifene, LY353381 (SERM3) and LY357489); steroidal estrogen receptor modulators (e.g. ICI-182,780). In some embodiments, the estrogen receptor modulators include selective estrogen receptor modulators (SERMs). The estrogen receptor modulator may be selected from the group consisting of: tamoxifen, toremifene, clomifene, or pharmaceutically acceptable salts and combinations thereof. In some embodiments, the estrogen receptor modulator is toremifene, or a pharmaceutically acceptable salt thereof.

Anti-inflammatory agents include any agent that counteracts or suppresses the inflammatory process. Anti-inflammatory agents, include, but are not limited to: steroids; non-steroidal anti-inflammatory agents (NSAIDs); glucocorticoid receptor blockers (e.g., mifepristone, and the like); and immunomodulators (e.g., Interferon Alfa-2A (Roferon-A), Interferon Alfa-2b (Intron-A), Interferon Alfa-2b and Ribavirin combo Pack (Rebetron), Interferon Alfa-N3 (Alferon N), Interferon Beta-1A (Avonex), Interferon Beta-1B (Betaseron), Interferon Gamma, and the like). In some embodiments, the anti-inflammatory agent is melatonin.

The estrogen receptor modulator, anti-inflammatory agent, or pharmaceutically acceptable salts thereof, may be administered simultaneously, either in a single composition, or as two distinct compositions using the same or different administration routes, or sequentially in any order. When administered sequentially, any time interval (e.g., 5 min, 10 min, 30 min, 1 hour, 2 hours, 4 hours, 6 hours, 12 hours) may separate the administration of the estrogen receptor modulator and the anti-inflammatory agent.

The estrogen receptor modulator, anti-inflammatory agent, or pharmaceutically acceptable salts thereof, may be administered to subjects by a variety of methods. In any of the uses or methods described herein, administration can be by various routes known to those skilled in the art, including without limitation oral, inhalation, intravenous, intramuscular, topical, subcutaneous, systemic, and/or intraperitoneal administration to a subject in need thereof. In select embodiments, the estrogen receptor modulator, anti-inflammatory agent, or pharmaceutically acceptable salts thereof, are administered orally.

The amount of the compound of the estrogen receptor modulator, anti-inflammatory agent, or pharmaceutically acceptable salts thereof, required for use in treatment will vary not only with the particular compound or salt selected but also with the route of administration, the nature and/or symptoms of the viral infection and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein.

In some embodiments, the estrogen receptor modulator, anti-inflammatory agent, or pharmaceutically acceptable salts thereof, or compositions comprising thereof may be administered by oral administration or intravenous administration. In general, a suitable dose will often be in the range of from about 0.01 mg/kg to about 100 mg/kg, such as from about 0.05 mg/kg to about 10 mg/kg. For example, a suitable dose may be in the range from about 0.10 mg/kg to about 7.5 mg/kg of body weight per day, such as about 0.10 mg/kg to about 0.50 mg/kg of body weight of the recipient per day, about 0.10 mg/kg to about 1.0 mg/kg of body weight of the recipient per day, about 0.15 mg/kg to about 5.0 mg/kg of body weight of the recipient per day, about 0.2 mg/kg to 4.0 mg/kg of body weight of the recipient per day. The estrogen receptor modulator, anti-inflammatory agent, or pharmaceutically acceptable salts thereof, may be administered in unit dosage form; for example, containing 1 to 500 mg, 1 to 200 mg, 20 to 200 mg, 1 to 100 mg, 10 to 100 mg, 20 to 100 mg, or 20 to 60 mg of active ingredient per unit dosage form.

In some embodiments, the estrogen receptor modulator, or pharmaceutically acceptable salts thereof, is administered in unit dosage form containing 60 mg of the estrogen receptor modulator per unit dosage form. In some embodiments, the 60 mg unit dosage form is administered once a day, thus the estrogen receptor modulator is administered at a concentration of about 60 mg/day. In select embodiments, the estrogen receptor modulator is toremifene administered in unit dosage form, containing 60 mg unit dosage form, once a day.

The anti-inflammatory agent, or pharmaceutically acceptable salts thereof, may be administered at a concentration of up to 2000 mg/day, up to 1000 mg/day, up to 500 mg/day, up to 100 mg/day. In select embodiments, the anti-inflammatory agent, or pharmaceutically acceptable salts thereof, is administered at a concentration of between 50 and 100 mg/day. In some embodiments, the anti-inflammatory agent, or pharmaceutically acceptable salts thereof, is administered in unit dose forms. The unit dose forms may comprise between 20 and 100 mg. In select embodiments, the anti-inflammatory agent, or pharmaceutically acceptable salts thereof, is administered twice a day in unit dose forms comprising between 20 and 100 mg of the active agent. In exemplary embodiments, the anti-inflammatory agent, or pharmaceutically acceptable salts thereof, is administered twice a day with two unit doses; a first unit dose comprising 40 mg and a second unit dose comprising 60 mg, or a first unit dose comprising 20 mg and a second unit dose comprising 40 mg.

The desired doses of the estrogen receptor modulator, anti-inflammatory agent, or pharmaceutically acceptable salts thereof, may be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration may vary depending upon the age, weight, and the severity of the infection. The determination of effective dosage levels, that is the dosage levels necessary to achieve the desired result, can be accomplished by one skilled in the art using routine methods, for example, human clinical trials, in vivo studies and in vitro studies. For example, useful dosages of the estrogen receptor modulator, anti-inflammatory agent, or pharmaceutically acceptable salts thereof, can be determined by comparing their in vitro activity, and in vivo activity in animal models.

Dosage amount and intervals may be adjusted individually to provide plasma levels of the active moiety which are sufficient to maintain the modulating effects, or minimal effective concentration (MEC). The MEC will vary but can be estimated from in vivo and/or in vitro data. Dosages necessary to achieve the MEC will depend on individual characteristics and route of administration. However, FIPLC assays or bioassays can be used to determine plasma concentrations. Dosage intervals can also be determined using MEC value. The attending physician will know how to adjust treatment to higher levels if the clinical response were not adequate (precluding toxicity).

The estrogen receptor modulator and/or the anti-inflammatory agent may be provided and administered as individual or combined pharmaceutical compositions or formulations which may include one or more pharmaceutically acceptable carriers. The term “pharmaceutically acceptable carrier,” as used herein, means a non-toxic, inert solid, semi-solid or liquid filler, diluent, binder, disintegrant, colorant, lubricant, preservatives, colvents, encapsulating material or formulation auxiliary of any type. Some examples of materials which can serve as pharmaceutically acceptable carriers are sugars such as, but not limited to, lactose, glucose and sucrose; starches such as, but not limited to, com starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compositions or formulations may be formulated for administration by, for example, solid dosing, injection, implants, or oral, buccal, sublingual, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage. The route or administration and the form of the composition will dictate the type of carrier to be used.

### 3. Combinations and Kits

Also within the scope of the present disclosure are combinations and kits. The combinations and kits may comprise a selective estrogen receptor modulator, or a pharmaceutically acceptable salt thereof and an anti-inflammatory agent, or a pharmaceutically acceptable salt thereof.

The term “combination” refers to either a fixed combination in one dosage unit formulation or composition, or as separate dosage formulations or compositions suitable for use together either simultaneously or sequentially within time intervals that especially allow that the combination to show a cooperative (e.g., synergistic) effect.

Individual member components of the combinations and kits may be physically packaged together or separately. The components of the combinations and kits may be provided in bulk packages (e.g., multi-use packages) or single-use packages. The components of the combinations and kits may be provided in pre-filled delivery devices, to be administered by the subject themselves or by another outside of a hospital or medical setting. The kits can also comprise instructions for using the components of the kit. The instructions are relevant materials or methodologies pertaining to the kit. The materials may include any combination of the following: background information, list of components, brief or detailed instructions for using or administering the components, potential warnings regarding the components (e.g. side effects), and any other related documents. Instructions can be supplied with the kit or as a separate member component, either as a paper form or an electronic form which may be supplied on computer readable memory device or downloaded from a website.

It is understood that the disclosed kits and combinations can be employed in connection with the disclosed methods. The kits and combinations provided herein are in suitable packaging. Suitable packaging includes, but is not limited to, vials, bottles, jars, flexible packaging, blister packs, and the like. The disclosed kits or combinations may further comprise shipping and/or packaging containers, wherein the components in their suitable packaging are present inside said shipping and/or packaging containers

### 4. Examples

### Example 1

### SARS-CoV-2 Virus-Host Interactome

Four host gene/protein sets were assembled for SARS-CoV-2: (1) SARS2-DEG, representing the differentially expressed genes (DEGs) from the transcriptomic data of SARS-CoV-2 infected primary human bronchial epithelial cells; (2) SARS2-DEP, representing the differentially expressed proteins (DEPs) from the proteomic data of SARS-CoV-2 infected human Caco-2 cells; (3) HCoV-PPI, representing the literature-based virus-host proteins across multiple human coronaviruses (HCoVs), including the SARS-CoV-1 (from the 2002-2003 pandemic) and MERS-CoV; and (4) SARS2-PPI (SARS-CoV-2 specific virus-host PPIs). Since HCoV-PPI and SARS2-PPI are both physical virus-host PPIs, those sets were combined as the fifth data set, PanCoV-PPI.

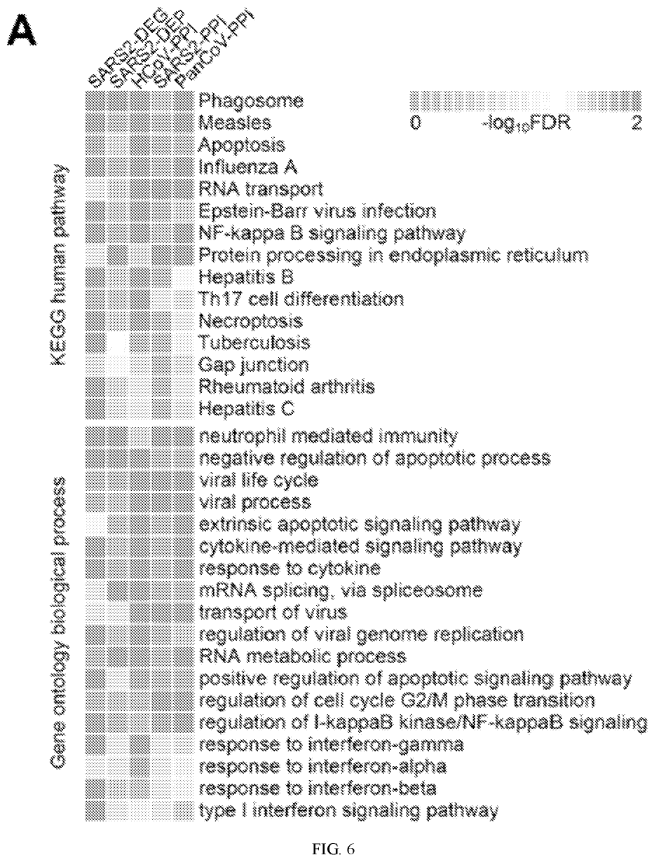

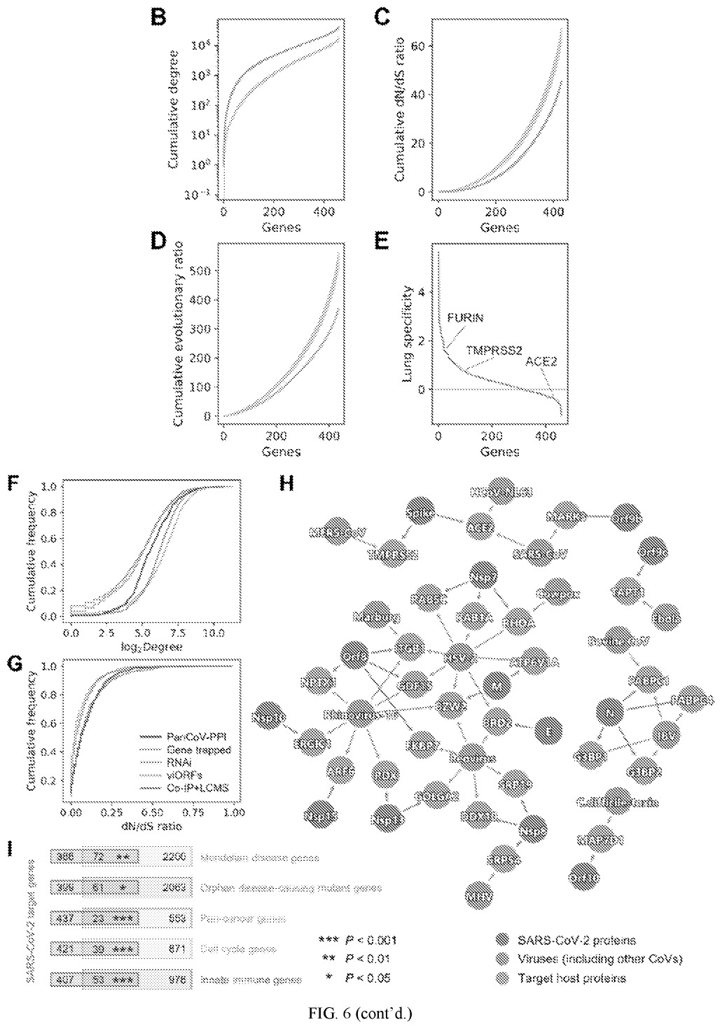

Functional enrichment analyses were performed for the five different data sets. These data sets shared several common pathways and ontology terms (FIG. 6A), such as phagosome, measles, apoptosis, NF-kappa B signaling pathway, neutrophil-related immunity, apoptotic processes, virus transport, viral genome replication, and response to interferon, they differed considerably in terms of their most significantly enriched pathways. This was especially noticeable for SARS2-DEP and SARS2-PPI. While SARS2-DEG and HCoV-PPI showed more enrichment in immune responses and viral pathways; SARS2-DEP was more related to various cellular metabolic pathways; and SARS2-PPI was more enriched in DNA replication, RNA transcription, and protein translation. These observations suggested that these different SARS-CoV-2 data sets capture complementary aspects of the biological and cellular states of the viral life cycle and host immunity. Therefore, building a global virus-host map (including interactome, transcriptome, and proteome) that incorporates data from transcriptomics, proteomics, and physical virus-host PPIs was key for a better understanding of the pathogenesis of COVID-19.

In addition to identifying the functions that these data sets represent, the network patterns (node degree in the human PPI network) and bioinformatics features of these SARS-CoV-2 data sets were characterized, including ratio of non-synonymous to synonymous substitutions (dN/dS), evolutionary rate ratio, and lung expression specificity (FIG. 6B-G). To find common as well as unique network and bioinformatic characteristics of SARS-CoV-2, four additional virus-host gene/protein networks identified by different methods for comparisons were also compiled: (1) 900 virus-host interactions connecting 10 other viruses and 712 host genes identified by gene-trap insertional mutagenesis, (2) 2,855 known virus-host interactions connecting 2,443 host genes and 55 pathogens identified from RNA interference (RNAi), (3) 579 host proteins mediating translation of 70 innate immune-modulating viral open reading frames (viORFs), and (4) 1,292 host genes mediating influenza-host interactions identified by co-immunoprecipitation and liquid chromatography-mass spectrometry (Co-IP+LC/MS). Host proteins in PanCoV-PPI (FIG. 6B) and four other data sets (SARS2-DEG, SARS2-DEP, HCoV-PPI, and SARS2-PPI) were more likely to be highly connected (high degree or connectivity) in the human PPI network, including several hubs, such as JUN, XPO1, MOV10, NPM1, VCP, and HNRNPA1. PanCoV-PPI had a comparable degree distribution with host proteins/genes identified by viORFs and Co-IP+LC/MS, although being marginally lower than that identified by RNAi and gene-trap insertional mutagenesis assay (FIG. 6F).

Expression patterns of genes in a specific disease-related tissue play crucial role for elucidation of disease pathogenesis and drug discovery. Given the major impact of SARS-CoV-2 on pulmonary function and lung injury, the lung-specific expression of genes in PanCoV-PPI were inspected using a Z score measure compared to other 30 tissues from the GTEx database. Most host genes for SARS-CoV-2 had high expression in lung (FIG. 6E) compared to other tissues; yet, ACE2 had a low expression in lung compared to other tissues. A recent study showed that ACE2 was primarily expressed in the epithelial cells in lungs, and only 3.8% of alveolar type 2 pneumocytes expressed both ACE2 and TMPRSS2, but it was significantly upregulated in smokers and 24-48 hours following SARS-CoV-2 infection. Another study also showed that despite relatively low expression of ACE2 in the lung, ACE2 was expressed in multiple epithelial cell types along the airway.

To inspect the evolutionary factors underlying the SARS-CoV-2-human PPIs, the selective pressure was investigated and evolutionary rates were quantified by the non-synonymous versus synonymous substitution rate ratios (dN/dS ratios, see Methods) using human-mouse orthologous gene pairs. PanCoV-PPI had a stronger purifying selection (lower dN/dS ratios (FIG. 6C) and evolutionary rate ratio (FIG. 6D)) compared to the same number of random genes. PanCoV-PPI was also comparable to other four virus-host genes/proteins identified by different assays (FIG. 6F and 6G) in terms of node degrees and dN/dS ratios. Altogether, these observations suggest that the virus-host PPIs assembled here offer a high-quality interactome map for SARS-CoV-2 for identifying pathogenesis and potential treatments for COVID-19.

To inspect shared viral pathways across different viruses and SARS-CoV-2, network overlap analysis of PanCoV-PPI was performed with 712 host genes across 10 types of other viruses identified by gene-trap insertional mutagenesis assays. A significant overlap of the SARS-CoV-2 host proteins with non-coronaviruses was found (P<0.002, Fisher's exact test, FIG. 6H). For example, BRD2, a transcriptional regulator which belongs to Bromodomain and Extra-Terminal motif family, was connected to SARS-CoV-2 and two other viruses, herpes simplex virus 2 (HSV-2) and reovirus. RHOA, encoding a small GTPase protein in the Rho family of GTPases, was connected to SARS-CoV-2, cowpox, and HSV-2 as well. RHOA has been reported to be involved in multiple human diseases, including cardiovascular disease and cancer. These observations indicated possible disease manifestations associated with SARS-CoV-2.

### Example 2

### SARS-CoV-2 Cellular Network Perturbations of Disease Manifestations

The overlap between SARS-CoV-2 host genes/proteins and the susceptibility gene sets implicated in different diseases and biological events was investigated (FIG. 6I). Host genes/proteins targeted by SARS-CoV-2 were significantly enriched in Mendelian disease (P=0.002, Fisher's exact test), orphan disease (P=0.044), and cancer (P<0.001). Mechanistically, SARS-CoV-2 target host genes were significantly enriched in cell cycle genes (P<0.001) and innate immune genes (P<0.001).

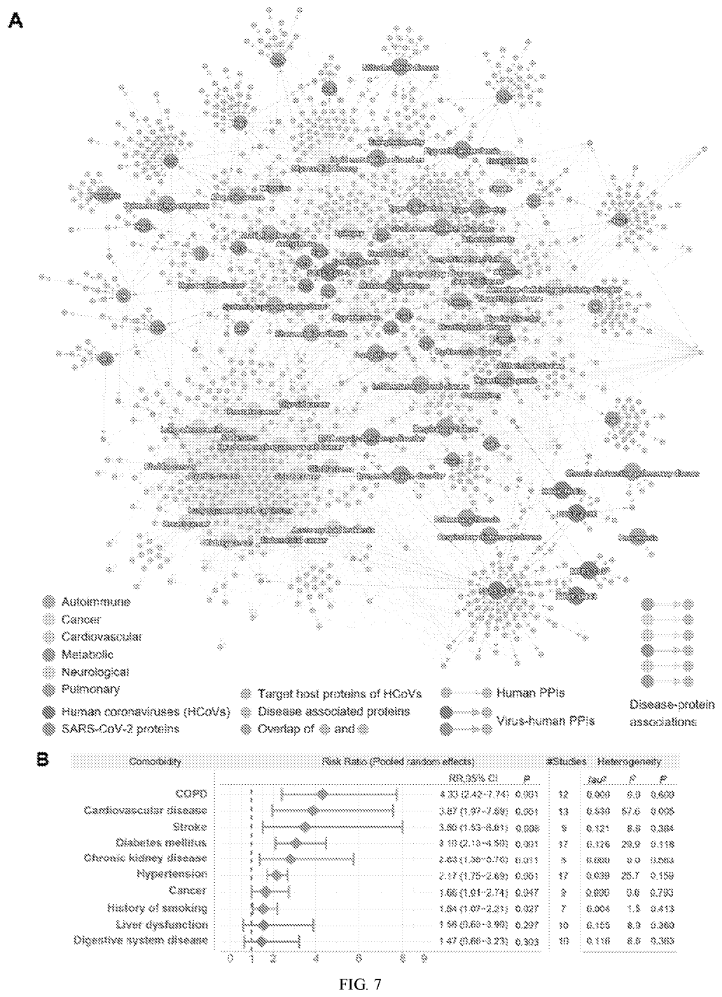

Potential COVID-19 comorbidities were identified by assembling the disease-protein network of COVID-19 and six disease categories. For cancer, the driver genes for pan-cancer and individual cancer types were retrieved from the Cancer Gene Census and a previous study. For autoimmune, pulmonary, neurological, cardiovascular, and metabolic categories, the associated genes/proteins were extracted from the Human Gene Mutation Database. Using the disease-protein network together with the SARS2-PPI (SARS-CoV-2 virus-host interactome) and HCoV-PPI (HCoV-host interactome) sets, the overall connectivity was examined in the human PPI network (FIG. 7A). To build the global network for the disease comorbidities, the PPIs were extracted from the human interactome for the virus target proteins and disease-associated proteins. Each small node indicates a virus target host protein (blue) or a disease-associated protein (green). Some disease-associated proteins can be directly targeted by the viruses, as shown in orange. Edges among these protein nodes indicate PPIs. Due to the tendency of having common disease-associated proteins, some disease categories tended to cluster closely, e.g., cancer and neurological. Diseases from other categories, such as autoimmune and pulmonary, were scattered. Most of the virus target proteins were connected with the disease-associated proteins, which suggested shared pathobiological pathways of COVID-19 and these diseases. Various cancer types formed a relatively distant module from the virus targets, compared to other disease categories.

Shown in FIG. 7A, these diseases can be targeted directly or interact with the targets of SARS-CoV-2 or other HCoVs. For example, among the four chronic obstructive pulmonary disease (COPD) associated proteins shown in the network, TGFB1 was a direct target of HCoV-229E, and all four proteins (TGFB1, DEFB1, SNAI1, and ADAM33) interacted with at least one SARS-CoV-2 viral protein target. The risk of various cardiovascular diseases was found to be increased in COVID-19 patients, including heart block, coronary artery disease, and congestive heart failure, and arrhythmia, which is consistent with clinically reported myocardial injury and cardiac arrest. These observations revealed common network relationship between COVID-19 and human diseases (FIG. 7A). Meta-analysis of 34 COVID-19 clinical studies were performed to evaluate the pooled risk ratios of 10 comorbidities among 4,973 COVID-19 positive patients (including 2,268 mild and 731 severe patients). The random effects model was used to estimate the pooled risk ratio of disease severity. The tau² and I² statistics were used to evaluate the heterogeneity among studies. 8 comorbidities had significantly higher risks in severe COVID-19 patients (FIG. 7B). The overall pooled risk ratio of COPD was 4.33 (95% confidence interval (CI) 2.42-7.74, P=0.001) in 12 low heterogeneous clinical studies (I²=0.0%, P=0.6). The COVID-19 patients with cardiovascular diseases had a risk ratio of 3.87 (95% CI 1.97-7.59, P=0.001), and there was a slightly higher heterogeneity across the 13 studies (I²=57.6%, P=0.005). Patients with stroke, diabetes, chronic kidney disease, hypertension, cancer, and history of smoking were also found to have higher risk ratios of severe COVID-19.

### Example 3

### Network-Based Measure of COVID-19-Associated Disease Manifestations

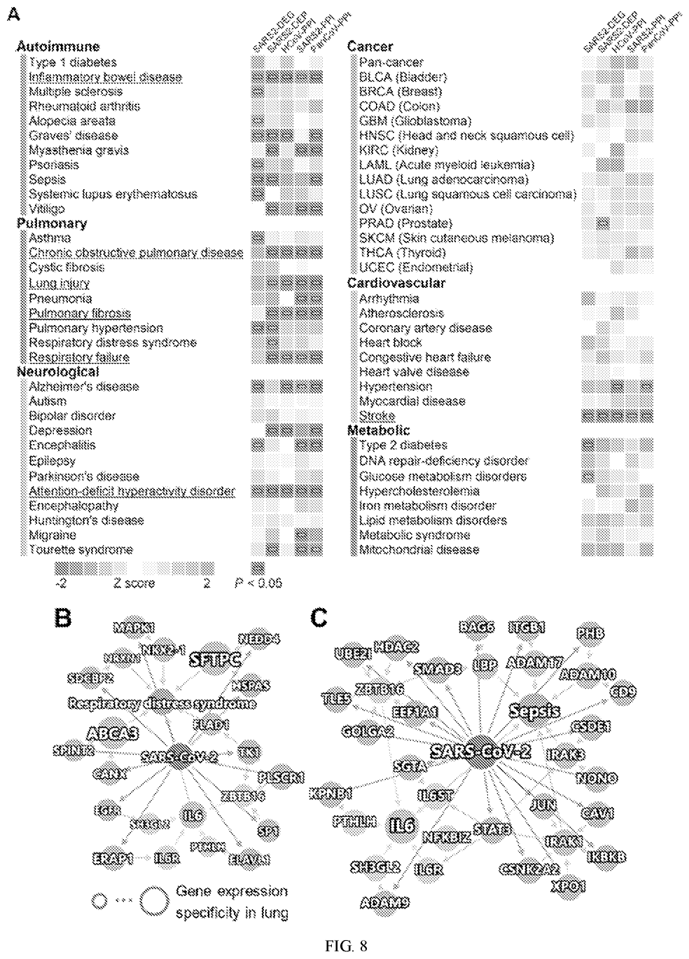

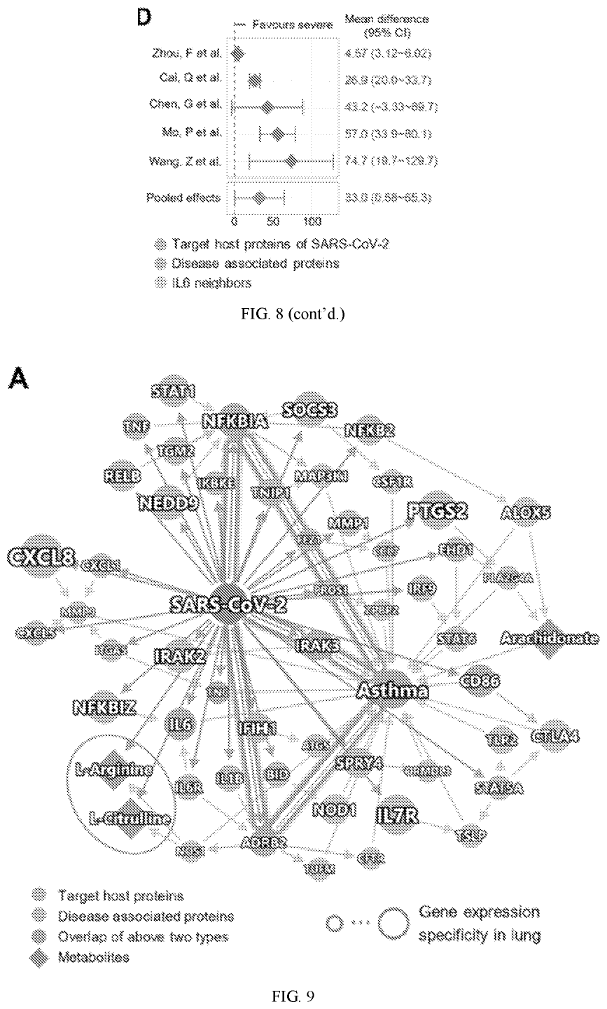

The network-based relationships of the 64 diseases across the 6 categories to COVID-19 were systematically evaluated (FIG. 8A). State-of-the-art network proximity measure was used to evaluate the connectivity and the closeness of the disease proteins and SARS-CoV-2 host proteins, taking the topology of the human interactome network into consideration. To test the significance of the proximity, Z scores and P values were calculated based on the permutation tests and are shown in FIG. 8A. Each disease-disease pair had a well-defined network-based footprint. If the footprint between COVID-19 module and another disease module was significantly close (low Z score and P<0.05), the magnitude of the proximity was indictive of their biological relationship: closer network proximity (FIG. 8A) of SARS-CoV-2 host genes/proteins with a disease module indicates higher potential of manifestation between COVID-19 and a specific disease. Immunological, pulmonary, and neurological diseases showed significant proximity to SARS-CoV-2 data sets more frequently than do cancer, cardiovascular, and metabolic diseases. Some diseases had significant proximities to more than one SARS-CoV-2 data sets, most notably inflammatory bowel disease (IBD), attention-deficit/hyperactivity disorder, and stroke, which achieved significant P value for all five SARS-CoV-2 protein sets. Pulmonary diseases, including COPD, lung injury, pulmonary fibrosis, and respiratory failure, achieved four significant proximities. Some diseases had significant proximities to certain SARS-CoV-2 data sets, indicating associations at certain levels, e.g., asthma (transcriptomic), respiratory distress syndrome (proteomic), and hypertension.

Network visualization further showed the connections between SARS-CoV-2 and other diseases, for example, respiratory distress syndrome (FIG. 8B), sepsis (FIG. 8C), and COPD. Multiple SARS-CoV-2 host proteins were directly connected with the disease-associated proteins (FIG. 8B). ABCA3 is a lipid transporter located in the outer membrane of lamellar bodies in AT2 cells; the mutations of the ABCA3 gene can disrupt pulmonary surfactant homeostasis and lead to inherited pulmonary diseases. Another membrane surface protein, the pulmonary-associated surfactant protein C encoded by SFTPC, can cause lung injury when misfolded. For sepsis, several inflammatory and immune-related proteins, such as IRAK1, IRAK3, IKBKB, and STAT3, suggested overlap of the inflammatory response activated in COVID-19 and sepsis (FIG. 8C). It has been reported that an overzealous production of certain cytokines, such as IL-6, caused by dysregulation of innate immune responses to SARS-CoV-2 infection can result in a ‘cytokine storm’, better known as cytokine release syndrome (CRS). The potential prognoses of acute respiratory distress syndrome and sepsis using IL-6 expression levels were also established. IL-6 had a significantly increased expression level in the human bronchial epithelial cells infected with SARS-CoV-2 from the SARS2-DEG data set. In addition, it was also potentially affected by SARS-CoV-2 through multiple PPIs (FIG. 8B and 8C, IL-6 neighbors), such as IL-6R, endophilin Al (encoded by SH3GL2), and parathyroid hormone like hormone (encoded by PTHLH). The random effects meta-analysis of 5 clinical studies of COVID-19 revealed that there was an increase of IL-6 levels in severe COVID-19 patients compared to non-severe COVID-19 patients (FIG. 8D). The mean difference was 33.0 pg/ml (95% CI 0.58-65.3, FIG. 8D) with high literature heterogeneity (I²=94%, P<0.001). These results indicated that IL-6 plays a critical role in COVID-19-associated respiratory distress syndrome and sepsis. IL-6 antagonists including tocilizumab (NCT04315480) and sarilumab (NCT04327388) are being investigated in clinical trials for treatment of patients with severe COVID-19.

### Example 3

### Inflammatory Endophenotypes Shared by COVID-19 and Asthma

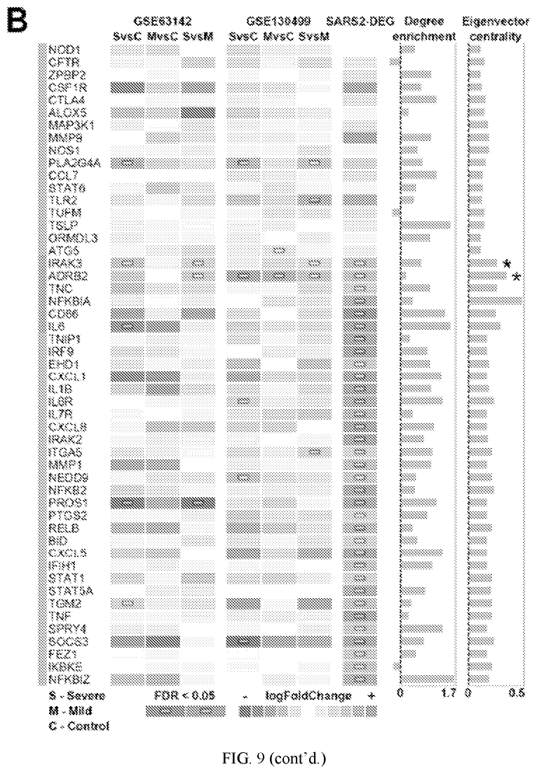

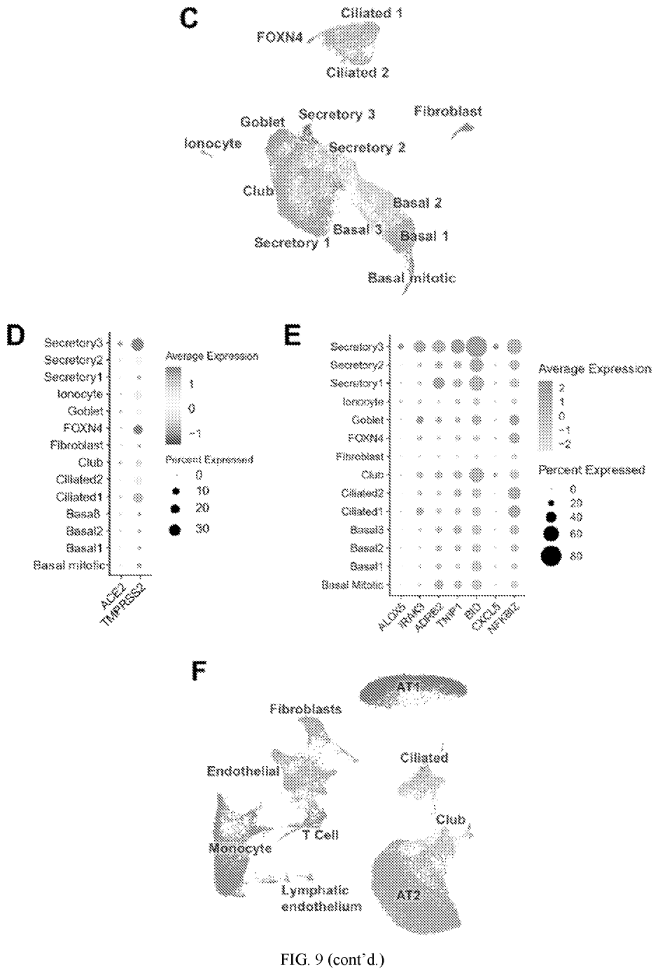

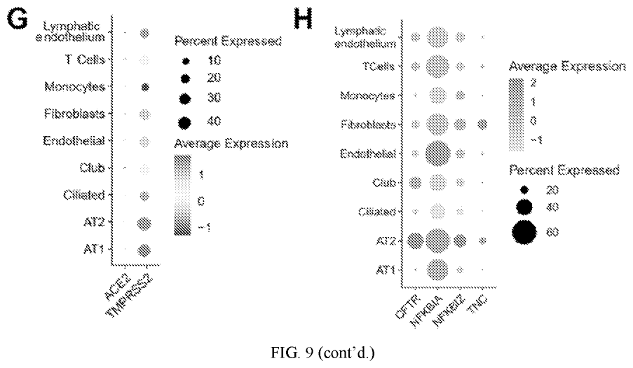

Patients with severe COVID-19 symptoms showed a higher prevalence of dyspnea (P<0.001). To understand associations between COVID-19 and respiratory disease (including asthma), a multi-modal analysis utilizing metabolomics and transcriptomics data from two previous asthma cohorts (see Methods) under the human interactome network model was adopted. FIG. 9A shows the subnetwork of the connections among SARS-CoV-2 target host proteins and asthma-associated proteins. Most of these proteins had enriched connections within the subnetwork (FIG. 9B, blue bars) (more connections in the subnetwork than in a random network of the same size in the human interactome, see Methods). Six overlapping proteins (orange) from both groups were identified: NFKBIA, IRAK3, TNC, IL6, ADRB2, and CD86. A recent study showed that glucose metabolism plays a key role in influenza A-regulated cytokine storm. The unique plasma metabolome of asthmatics versus healthy controls also suggested activated inflammatory and immune pathways. Therefore, in addition to PPIs, metabolomics data generated in a previously assembled asthma cohort was integrated. By matching the enzymes of the differential metabolites and the proteins in the PPI network, three key metabolites were found, including arachidonate, L-arginine, and L-citrulline. L-arginine and L-citrulline were decreased in the sera of COVID-19 patients. A previous study showed that these metabolites were also decreased in asthma patients. Arachidonate, the precursor of a variety of products that regulate inflammatory pathways, was found to have an increased level in the inflamed airways of asthmatics. Arachidonate can be converted by 5-lipoxygenase encoded by ALOX5 to leukotriene, which is release during an asthma attack and is responsible for the bronchoconstriction.

The DEGs from two asthma cohorts from the Severe Asthma Research Program were examined. Utilizing two bulk RNA-Seq data sets from asthma patients and healthy controls, elevated expression of IRAK3 and ADRB2 in SARS-CoV-2 infected human bronchial epithelial cells was identified (FIG. 9B). IRAK-M, encoded by IRAK3, regulates the toll-like receptor/interleukin (IL)-1 receptor pathway and NF-κB pathway, and IRAK3 was identified as an asthma susceptibility gene. ADRB2 encodes the beta 2-adrenergic receptor. The polymorphisms of ADRB2 (p.Arg16Gly and p.Gln27Glu) increased the risk of asthma occurrence, and p.Gln27Glu was associated with asthma severity. Altogether, altered IRAK3 and ADRB2 may explain relationships between COVID-19 and asthma, though these findings require experimental and clinically validation in patients with these disorders.

To understand expressions of the proteins in the asthma-COVID-19 network across different cell types, especially those cells which express ACE2, the single-cell RNA-Seq data from bronchial epithelium (FIG. 9C) and lung (FIG. 9F) was analyzed. Consistent with previous studies, ACE2 and TMPRSS2 had a relatively higher expression in a subtype of the secretory cells (secretory 3 cells), compared to other bronchial epithelial cell types (FIG. 9D). In the lung, ACE2 and TMPRSS2 had a relatively higher expression in AT2 cells (FIG. 9G). The expression of the genes in the asthma-COVID-19 network (FIG. 9A) was further examined in these cell types. Several genes were also more highly expressed in secretory 3 cells (FIG. 9E, ALOX5, IRAK3, ADRB2, TNIP1, BID, CXCL5, and NFKBIZ) and in AT2 cells (FIG. 9H, CFTR, NFKBIA, NFKBIZ, and TNC), than in other cell types. IRAK3 and ADRB2 were among the six overlapped genes, potentially implicating IRAK3 and ADRB2 in COVID-19-associated asthma at the single cell level as well.

### Example 4

### Immune Pathobiology Shared by COVID-19 and Inflammatory Bowel Disease

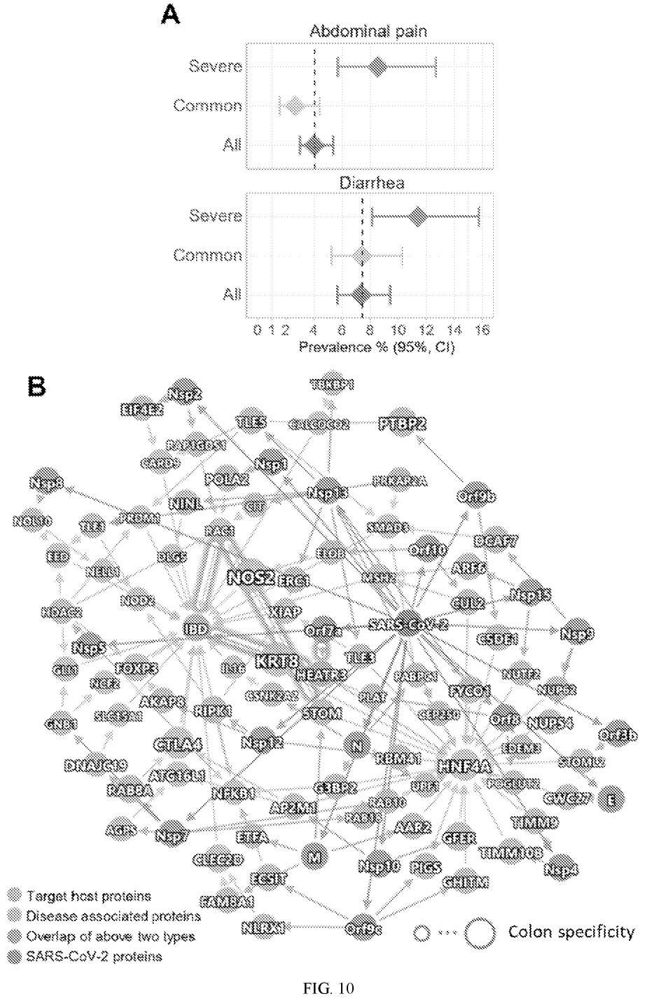

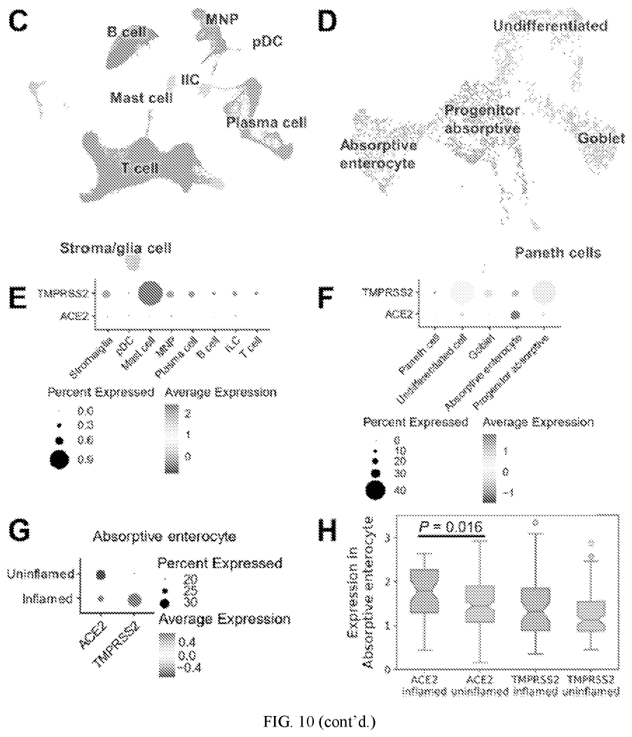

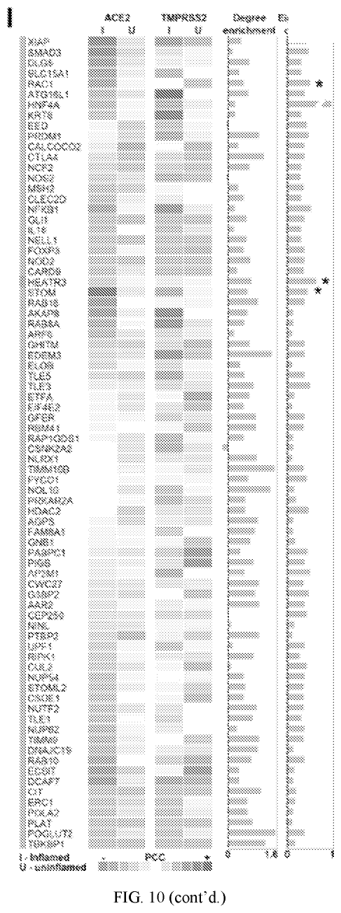

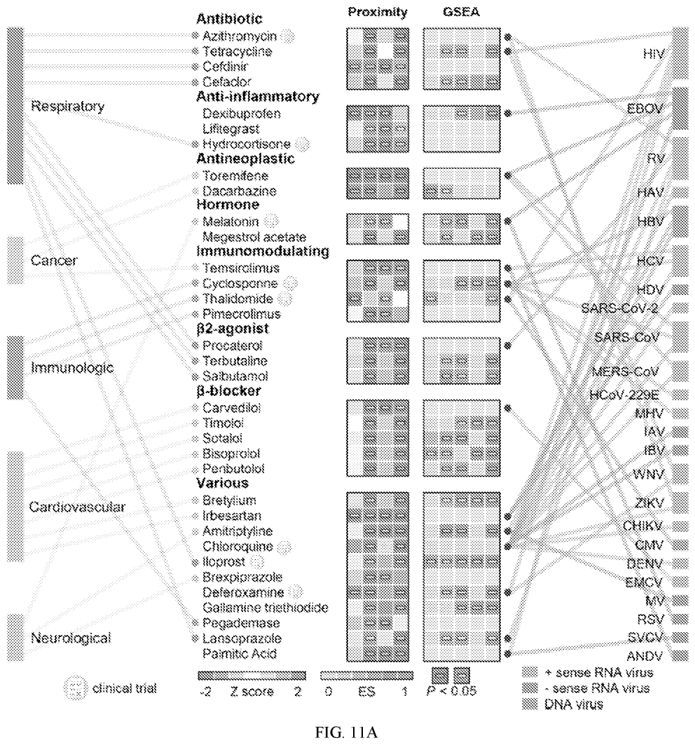

It has been shown that human small intestine is an additional SARS-CoV-2 target organ using confocal- and electron-microscopy. Diarrhea is now well-described as an occasional presenting symptom of COVID-19. The network proximity analysis showed a significant association of COVID-19 and IBD across all five SARS-CoV-2 data sets (FIG. 8A). In addition, severe COVID-19 patients had higher risks of abdominal pain and diarrhea (FIG. 10A). To understand these associations at the cellular level, network analysis was integrated with single-cell RNA-Seq analysis using publicly available data. As shown in FIG. 10B, although only one IBD-associated protein, HEATR3, was found to be the target of the SARS-CoV-2 protein Orf7a, other IBD-associated proteins showed enriched number of connections to the SARS-CoV-2 target proteins (FIG. 10I, blue bars).

Using single-cell data from the ileum (distal small bowel) in Crohn's disease patients, ACE2 and TMPRSS2 had low to undetectable expression in the non-epithelial cells (FIG. 10C and 10E). However, they showed higher expression levels in the epithelial cells, especially absorptive enterocytes (FIG. 10D and 10F). both ACE2 and TMPRSS2 had elevated expression levels in inflamed cells compared to uninflamed cells in the absorptive enterocytes (FIG. 10G). In absorptive enterocytes expressing ACE2, the expression of ACE2 was significantly increased (FIG. 10H, P=0.016) in the inflamed ileal tissues of Crohn's disease patients compared to uninflamed tissues. These observations prompted investigation of the co-expression of the network genes in the absorptive enterocytes (FIG. 10I). Several genes showed elevated co-expression with ACE2 in inflamed cells, including XIAP, SMAD3, DLG5, SLC15A1, RAC1, STOM, RAB18, and AKAP8.

SARS-CoV-2 protein Orf7a can directly interact with HEATR3, whose variant was shown to be associated with increased risks of IBD by genome-wide association study. Also, SARS-CoV-2 infection may impact RAC1 signal transduction pathways. RAC proteins play important roles in many inflammatory pathways, and their dysregulation can be pathogenic. Increased RAC1 expression by single nucleotide polymorphisms promotes an inflammatory response in the colon. Mercaptopurine, an effective treatment for IBD, was found to lower RAC1 expression in IBD patients. Since RAC1 and ACE2 had higher co-expression in inflamed enterocytes (FIG. 10I), it is highly possible that these inflamed cells are more susceptible to SARS-CoV-2 infection, and that the infection could lead to an altered RAC1 expression level through PPIs with virus target proteins STOM, HDAC2, POLA2, CIT, and RAP1GDS1 (FIG. 10B). Notably STOM was also highly co-expressed with ACE2 in inflamed cells compared to uninflamed cells (FIG. 10I).

### Example 5

### Network-Based Drug Repurposing for COVID-19

Knowledge of the complex interplays between SARS-CoV-2 targets and human diseases indicated possibilities of drug repurposing, as the drugs that target other diseases could potentially target SARS-CoV-2 through the shared functional PPI networks. Drug repurposing efforts may also reveal unrecognized biological connections between diseases that existing drugs treat and COVID-19. For example, the aforementioned anti-inflammatory drugs, tocilizumab and sarilumab that are now being tested for COVID-19 are originally used for rheumatoid arthritis. Although not significant, the network proximity results showed that rheumatoid arthritis had small network proximities (negative Z scores) across all five SARS-CoV-2 data sets (FIG. 8A). Another drug, the thiopurine mercaptopurine, which has been used to treat IBD, was one of the top repurposable drugs for COVID-19 proposed previously.

Therefore, network-based drug repurposing was performed using the existing knowledge of the drug-target network and the global map of the SARS-CoV-2 interactome built herein. The basis for the network-based drug repurposing methodologies rests on the observation that for a drug with multiple targets to be effective against a disease, its target proteins should be within or in the immediate vicinity of the corresponding subnetwork of the disease in the human interactome, as demonstrated in multiple diseases previously. Using the network proximity framework, the “closest” proximities of nearly 3,000 drugs and the four SARS-CoV-2 host gene/protein profiles (SARS2-DEG, SARS2-DEP, HCoV-PPI, and SARS2-PPI) was measured. Additionally, gene set enrichment analysis (GSEA) was performed using five gene/protein expression data sets, including one SARS-CoV-2 transcriptomics, one SARS-CoV-2 proteomics, one MERS-CoV and two SARS-CoV-1 transcriptomics data sets. GSEA was used to evaluate the individual drugs for their potential to reverse the expression at the transcriptome or proteome level altered by the viruses.

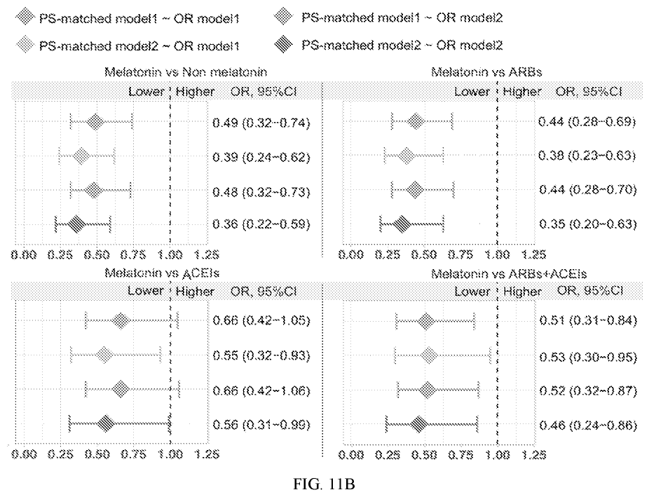

In total, 34 drugs were computationally identified as associated (Z<−1.5 and P<0.05, permutation test) with the SARS-CoV-2 data sets (SARS2-DEG, SARS2-DEP, HCoV-PPI, and SARS2-PPI). These drugs were significantly proximal to two or more SARS-CoV-2 host protein sets (FIG. 11A). The disease categories that these drugs have been used to treat are also shown in the figure. Ten drugs have been used to treat respiratory-related diseases, and the most common categories for these drugs were antibiotic and β2 agonist. The next most popular disease category was cardiovascular diseases, for which seven drugs were predicted. Among the 34 drugs, three drugs achieved significant network proximity with all four SARS-CoV-2 data sets investigated here. These drugs were antibiotic drug cefdinir, which is a cephalosporin for the treatment of bacterial infections; antineoplastic drug toremifene, a selective estrogen receptor modulator shows striking activities in blocking various viral infections at low micromolar levels, including Ebola virus (50% inhibitive concentration [IC₅₀]=˜1 μM), MERS-CoV (50% effective concentration [EC₅₀]=12.9 μM), SARS-CoV-1 (EC₅₀=11.97 μM), and SARS-CoV-2 (IC₅₀=3.58 μM); and antihypertensive drug irbesartan, an angiotensin receptor block that can inhibit viral entry by inhibiting sodium/bile acid cotransporters.

### Example 6

### Validating Drug-Outcome Relationships on COVID-19 Using Patient Data

Drug-outcome relationships were next evaluated using a large-scale patient data from the Cleveland Clinic COVID-19 patient registry. Subject matter expertise based on: (i) literature-reported antiviral effects for human coronaviruses or other types of human viruses; (ii) strength of network proximity prediction and GSEA analysis (FIG. 11A); and (iii) availability of sufficient patient data for meaningful evaluation (exclusion of infrequently used drugs) was used. Applying these criteria resulted in the identification melatonin, a physiologic hormone common to many living organisms.

Melatonin regulates human circadian rhythm, a homeostatic mechanism comprised of interacting cellular clocks and environmental influences rhythmically altering translation of genes responsible for melatonin-mediated anti-inflammatory and immune-related effects. To test clinical benefits of melatonin on COVID-19, a retrospective COVID-19 cohort analysis was performed to inspect the potential prevention effect of melatonin. Among a total of 18,118 patients tested for COVID-19 in the Cleveland Clinic Health System in Ohio and Florida, 1,675 patients were diagnosed with COVID-19 between March 8 and Apr. 16, 2020. Melatonin usage was found to be associated with a 50-60% reduced likelihood of a positive laboratory test result for SARS-CoV-2 (OR=0.36, 95% CI 0.22-0.59, FIG. 11B) after adjusting for age, sex, race, and various disease comorbidities (diabetes, hypertension, heart disease, and pulmonary conditions, and many others) using a propensity score (PS) matching method.

Angiotensin-converting enzyme inhibitors (ACEIs) and angiotensin II receptor blockers (ARBs) are two common types of drugs for treatment of hypertension. A recent study showed that inpatient use of ACEI/ARB was associated with lower risk of all-cause mortality compared with ACEI/ARB non-users hospitalized COVID-19 patients with hypertension. An observational study for three cohorts was performed using user active comparator design and PS adjustment for confounding factors as described previously. Melatonin usage was associated with reduced risk of the likelihood of a positive laboratory test result for SARS-CoV-2 compared to ARBs (OR=0.35, 95% CI 0.20-0.63) and ACEIs (OR=0.56, 95% CI 0.31-0.99), and the pooled data of ARBs and ACEIs (OR=0.46, 95% CI 0.24-0.86) (FIG. 11B). Without being bound by theory, exogenous melatonin may boost host immunity and inhibit NLRP3 Inflammasome.

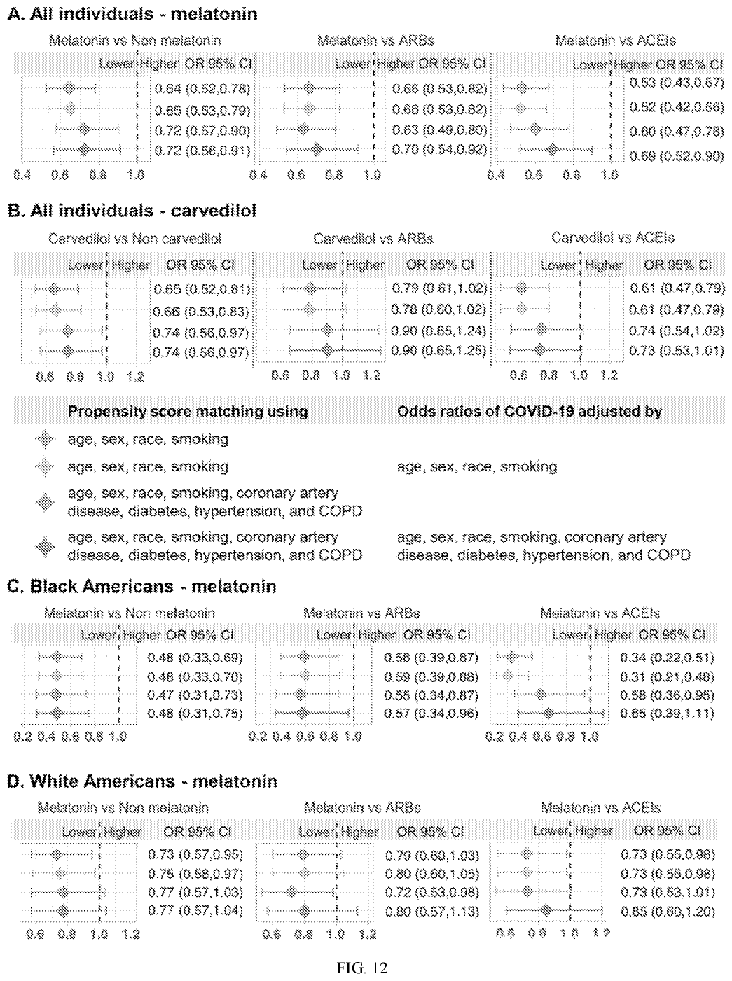

A retrospective COVID-19 cohort analysis was conducted to validate the potential prevention effect of melatonin and carvedilol (FIGS. 12A and 12B) among a total of 26,779 patients tested for COVID-19 in the Cleveland Clinic Health System in Ohio and Florida, of which 8,274 patients were diagnosed as SARS-CoV-2 positive as confirmed by reverse transcription—polymerase chain reaction (RT-PCR) between Mar. 8 and Jul. 27, 2020.

Melatonin usage was associated with a 28% reduced likelihood of a positive laboratory test result for SARS-CoV-2 (odds ratio [OR]=0.72, 95% CI 0.56-0.91; FIG. 12A) after adjusting for age, sex, race, smoking history, and various disease comorbidities (diabetes, hypertension, coronary artery disease, and COPD) using a propensity score (PS) matching method. Angiotensin-converting enzyme inhibitors (ACEIs) and ARBs are 2 common types of drugs for treatment of hypertension.

A recent study showed that inpatient use of ACEI/ARB was associated with lower risk of all-cause mortality compared with ACEI/ARB non-use among hospitalized COVID-19 patients with hypertension. Several recent studies also showed that there was no association of ARBs and ACEIs with the risk of SARS-CoV-2 infection. An observational study for 3 cohorts was performed using user active comparator design with ARBs and ACEIs used as comparators and PS adjustment for confounding factors as described. Melatonin usage was significantly associated with a reduced likelihood of a positive laboratory test result for SARSCoV-2 compared to use of ARBs (OR=0.70, 95% CI 0.54-0.92) and ACEIs (OR=0.69, 95% CI 0.52-0.90) after adjusting for age, sex, race, smoking history, and various disease comorbidities (FIG. 12A). Altogether, network-based prediction and multiple observational analyses (FIG. 12A) suggested that melatonin usage offers a potential prevention and treatment strategy for COVID-19.

Carvedilol use was significantly associated with a reduced likelihood of a positive laboratory test result for SARS-CoV-2 (OR=0.74, 95% CI 0.56-0.97) after adjusting for age, sex, race, smoking history, and various disease comorbidities (FIG. 12B). Yet, carvedilol did not show a significant advantage compared to ARBs (OR=0.90, 95% CI 0.65-1.25) or ACEIs (OR=0.73, 95% CI 0.53-1.01).

To test whether a clinically meaningful effect can be observed in different subgroups of patients, 5 different subgroups were generated: asthma patients, hypertension patients, diabetes patients, black Americans (African Americans), and white Americans. Melatonin was significantly associated with a 52% reduced likelihood of a positive laboratory test result for SARS-CoV-2 in black Americans (OR=0.48, 95% CI 0.31-0.75; FIG. 12C) after adjusting for age, sex, race, smoking, and various disease comorbidities, which is stronger than the association in white Americans (OR=0.77, 95% CI 0.57-1.04; FIG. 12D). In addition, in black Americans, melatonin usage was significantly associated with a reduced likelihood of a positive laboratory test result for SARSCoV-2 compared to ARB usage (OR=0.57, 95% CI 0.34-0.96; FIG. 12C), while there was no significant difference compared to ACEI usage (OR=0.65, 95% CI 0.39-1.11; FIG. 12C). Yet, melatonin usage was not significantly associated with a reduced likelihood of a positive laboratory test result for SARS-CoV-2 compared to use of ARBs (OR=0.80, 95% CI 0.57-1.13; FIG. 12D) and ACEIs (OR=0.85, 95% CI 0.60-1.20; FIG. 12D) in white Americans. Among the 3 comorbid disease subgroup analyses, melatonin usage was significantly associated with a reduced risk of SARS-CoV-2 positive test in diabetes patients only (OR=0.52, 95% CI 0.36-0.75); there was no significant association for asthma (OR=0.61, 95% CI 0.36-1.06) or hypertension (OR=0.80, 95% CI 0.61-1.05) patients.

### Example 7

### Materials and Methods

Building the data sets of SARS-CoV-2 target host genes/proteins. Four SARS-CoV-2 data sets of target host genes/proteins were assembled: (1) 246 differentially expressed genes in human bronchial epithelial cells infected with SARS-CoV-2 (GSE147507), denoted as SARS2-DEG; (2) 293 differentially expressed proteins in human Caco-2 cells infected with SARS-CoV-2, denoted as SARS2-DEP; (3) 134 strong literature evidence-based pan-human coronavirus target host proteins from a recent study with 15 newly curated proteins, denoted as HCoV-PPI; (4) 332 proteins involved in the protein-protein interactions with 26 SARS-CoV-2 viral proteins identified by affinity purification-mass spectrometry, denoted as SARS2-PPI. Finally, due to the interactome nature of HCoV-PPI and SARS2-PPI, these data sets were combined as the fifth SARS-CoV-2 data set, which has 460 proteins and is denoted as PanCoV-PPI.

Building the disease gene profiles. The disease-associated gene sets were compiled from various sources. All databases were accessed on Mar. 26, 2020.

Cancer. The pan-cancer driver genes were retrieved from the Cancer Gene Census. Driver genes for individual cancer types were from a previous study.

Mendelian disease genes (MDGs). A set of 2,272 MDGs were retrieved from the Online Mendelian Inheritance in Man (OMIM) database.

Orphan disease-causing mutant genes (ODMGs). A set of 2,124 ODMGs were retrieved from a previous study.

Cell cycle genes. 910 human cell cycle genes were downloaded from a previous study identified by a genome-wide RNAi screening.

Innate immune genes. 1,031 human innate immunity genes were collected form InnateDB.

Genes associated with autoimmune, pulmonary, neurological, cardiovascular, and metabolic diseases. The disease-associated genes/proteins were extracted from the Human Gene Mutation Database (HGMD). A systematic search was manually performed for each disease using keywords such as disease name, alias, and symptoms to identify the disease terms in the HGMD. All disease terms were then verified for their relevance.

Functional enrichment analysis. Kyoto Encyclopedia of Genes and Genomes (KEGG) and Gene Ontology (GO) biological process enrichment analyses were performed to reveal the biological relevance and functional pathways of the five SARS-CoV-2 data sets. All functional enrichment analyses were performed using Enrichr.

Selective pressure and evolutionary rates characterization. The dN/dS ratio and the evolutionary rate ratio was calculated as described previously (F. Cheng, et al., Molecular Biology and Evolution 31, 2156-2169 (2014)).

Tissue specificity analysis. The RNA-Seq data (transcripts per million, TPM) of 31 tissues from the GTEx V8 release (accessed on Mar. 31, 2020) were downloaded. Genes with count per million (CPM)≥0.5 in over 90% samples in a tissue were considered tissue-expressed genes and otherwise as tissue-unexpressed. To quantify the expression specificity of gene i in tissue t, the mean expression Eᵢ and the standard deviation σᵢ of a gene's expression were calculated across all considered tissues. The significance of gene expression specificity in a tissue was defined as:

$${z}_{it} = \frac{{E}_{it} - {E}_{i}}{{σ}_{i}} \qquad ( 1 )$$

Risk ratio analysis for COVID-19 patients. PubMed, Embase and Medrxiv databases were searched for publications as of Apr. 25, 2020. The search was limited to English articles describing the demographic and clinical features of SARS-CoV-2 cases. The search term (“SARS-COV-2” OR “COVID-19” OR “nCoV 19” OR “2019 novel coronavirus” OR “coronavirus disease 2019”) AND (“clinical characteristics” OR “clinical outcome” OR “comorbidities”) were used. Only research articles were included; reviews, case reports, comments, editorials, and expert opinions were excluded. Three criteria were used to select studies from a total of 1,054 initial hits: (1) studies that have ≥20 COVID-19 patients; (2) studies that grouped the outcomes with varying degree of severity of COVID-19 (e.g., severe vs. non-severe) according to the American Thoracic Society guidelines for community-acquired pneumonia; and (3) studies that were from different institutions. Two criteria were used for exclusion: (1) studies that focus on specific populations (e.g., only death cases, pregnant women, children or from family clusters); and (2) basic molecular biology research. Finally, 34 studies meeting these criteria were used for further analyses.

Random effects meta-analysis was performed to estimate the pooled risk ratio with 95% CI of 10 comorbidities between severe versus non-severe COVID-19 patients. Mantel-Haenszel method was used to estimate the pooled effects of results. DerSimonian-Laird method was used to estimate the variance among studies. The continuous data such as IL-6 levels were transformed to mean and standard deviations first using Wan's approach based on sample size, median and interquartile range. Next, inverse variance method was used to estimate the pooled mean difference and estimated the variance among studies by DerSimonian-Laird method. The pooled prevalence of 3 COVID-19 symptoms (abdominal pain, diarrhea, dyspnea) and 1 comorbidity (COPD) was estimated in three COVID-19 patient groups (severe, non-severe, and all) respectively. Random intercept logistic regression model was used to estimate pooled prevalence and maximum-likelihood estimator was used to quantify the heterogeneity of studies. The tau² and I² statistics were calculated for the heterogeneity among studies. I²<50% was considered low heterogeneity among studies, 50%<I²<75% was considered moderate heterogeneity, and I²>75% was considered high heterogeneity. All meta-analyses were conducted by meta and dmetar packages in the R v3.6.3 platform.

Building the human protein-protein interactome. A total of 18 bioinformatics and systems biology databases were assembled to build a comprehensive list of human PPIs with five types of experimental evidences: (1) binary, physical PPIs from protein three-dimensional (3D) structures; (2) binary PPIs tested by high-throughput yeast-two-hybrid (Y2H) systems; (3) kinase-substrate interactions by literature-derived low-throughput or high-throughput experiments; (4) signaling network by literature-derived low-throughput experiments; and (5) literature-curated PPIs identified by affinity purification followed by mass spectrometry (AP-MS), Y2H, or by literature-derived low-throughput experiments. Inferred PPIs based on gene expression data, evolutionary analysis, and metabolic associations were excluded. Genes were mapped to their Entrez ID based on the NCBI database. The official gene symbols were based on GeneCards. The final human protein-protein interactome used in this study included 351,444 unique PPIs (edges or links) connecting 17,706 proteins (nodes).

Network proximity measure. The “closest” network proximity measure throughout. For two gene/protein sets A and B, their “closest” distance d_{AB} was calculated as:

$$〈 {d}_{A B} 〉 = \frac{1}{‖ A ‖ + ‖ B ‖} ( {\sum }_{a ∈ A} {min}_{b ∈ B} d ( a , b ) + {\sum }_{b ∈ B} {min}_{a ∈ A} d ( a , b ) ) \qquad ( 2 )$$

where d(a, b) is the shortest distance of a and b in the human interactome. To evaluate the significance, a permutation test was performed using randomly selected proteins from the whole interactome that were representative of the two protein sets being evaluated in terms of their degree distributions. The Z score was calculated as:

$${Z}_{{d}_{A B}} = \frac{{d}_{A B} - \overline{{d}_{r}}}{{σ}_{r}} \qquad ( 3 )$$

where d̅ᵣ̅ and aᵣ were the mean and standard deviation of the permutation test. All network proximity permutation tests in this study were repeated 1,000 times.

Network-based comorbidity analysis. To reveal potential COVID-19 comorbidities, the network proximity of the disease-associated proteins for each disease and the five SARS-CoV-2 data sets was computed. SARS-CoV-2 target proteins with a non-negative tissue specificity in lung were used in the computation. The degree enrichment for protein i in a subnetwork was calculated as:

$${e}_{i} = \frac{{d}_{i} / n}{{D}_{i} / N} \qquad ( 4 )$$

where dᵢ is the degree of i in the subnetwork, n is number of nodes in the subnetwork, Dᵢ is the degree in the complete human protein interactome, and N is the total number of nodes in the interactome.

Bulk and single-cell RNA-Seq data analysis. Bulk RNA-Seq data sets for asthma patients were retrieved from the NCBI GEO database using the accession number GSE63142 and GSE130499. Differential expression of three comparisons, severe vs. control, mild vs. control, and severe vs. mild were performed using the GEO2R function. All single-cell data analyses and visualizations were performed with the R package Seurat v3.1.4. GSE134809 was downloaded from the NCBI GEO database. This data set contains 67,050 inflamed and uninflamed cells from the ileal samples of 8 patients with Crohn's disease. Single-cell data of normal lung and primary human bronchial epithelial cells were downloaded from https://data.mendeley.com/datasets/7r2cwbw44m/1. These data sets contain 39,778 lung cells and 17,451 bronchial epithelial cells. Qualifying cells based on the criteria from the original paper were used for the analysis. “NormalizeData” was used to normalize the data. “FindIntegrationAnchors” and “IntegrateData” functions were used to integrate cells from different samples. UMAP was used as the dimension reduction method for visualization. Cell type markers from the original paper were used to label the cell types.

Building the drug-target network. To evaluate whether a drug is closely associated with SARS-CoV-2 target proteins in the human interactome, the drug-target interaction information was gathered from several databases: DrugBank database (v4.3), Therapeutic Target Database (TTD), PharmGKB database, ChEMBL (v20), BindingDB, and IUPHAR/BPS Guide to PHARMACOLOGY. The interactions that have binding affinities Ki, Kd, IC₅₀ or EC₅₀<10 μM and a unique UniProt accession number with “reviewed” status were included. The details for building the experimentally validated drug-target network can be found in recent studies (F. Cheng, et al. Nat Commun 9, 2691 (2018); F. Cheng, et al., Nat Commun 10, 1197 (2019); F. Cheng, et al., Nat Commun 10, 3476 (2019), incorporated herein by reference).

Network-based drug repurposing. The “closest” network proximity was computed as described before for 2,938 FDA approved or investigational drugs and the five SARS-CoV-2 data sets. For prioritization, the drugs were ranked by their distance to the data sets (D<2) and Z score (Z<−1.5) from the network proximity analysis. The antiviral profiles of the highlighted drugs were manually curated. COVID-19 related clinical trials were retrieved on May 10, 2020.

Gene set enrichment analysis (GSEA). The gene set enrichment analysis was conducted as described previously (Y. Zhou, et al., Cell Discov 6, 14 (2020), incorporated herein by reference) as an additional evidence for drug repurposing. Briefly, for each drug and coronavirus target gene set, an enrichment score was computed to indicate whether a drug can reverse the effect of SARS-CoV-2 at the transcriptome or proteome level. Gene expression profiles for the drugs were retrieved from the Connectivity Map (CMAP) database. Five gene sets were evaluated: (1) the differentially expressed genes in human bronchial epithelial cells infected with SARS-CoV-2 (GSE147507); (2) the differentially expressed proteins in human Caco-2 cells infected with SARS-CoV-2; (3, 4) two transcriptome data sets of SARS-CoV-1 infected samples from patient's peripheral blood (GSE1739) and Calu-3 cells (GSE33267) respectively; (5) the differentially expressed genes in MERS-CoV infected Calu-3 cells (GSE122876).

The enrichment score ES was calculated for up- and down- regulated genes separately first. The overall ES was calculated as:

$$E S = \{ \begin{matrix}E {S}_{u p} - E {S}_{d o w n} , & sgn ( E {S}_{u p} ) \neq sgn ( E {S}_{d o w n} ) \\ 0 , & else\end{matrix} \qquad ( 5 )$$

where ESᵤₚ and ES_{down} were calculated using a_{up/down} and b_{up/down} as:

$$a = {max}_{1 \leq j \leq s} ( \frac{j}{s} - \frac{V ( j )}{r} ) \qquad ( 6 )$$

$$b = {max}_{1 \leq j \leq s} ( \frac{V ( j )}{r} - \frac{j - 1}{s} ) \qquad ( 7 )$$

j=1,2, . . . , s were the genes in the gene sets sorted in ascending order by their rank in the drug profiles. V(j) indicates the rank of gene j, where 1≤V(j)≤r, with r being the number of genes from the drug profile. Then, ES_{up/down} was set to a_{up/down} if a_{up/down}>b_{up/down}, and was set to −b_{up/down} if b_{up/down}>a_{up/down} Permutation test was performed to evaluate the significance. Drugs were considered to have potential treatment effect if ES>0 and P<0.05.

Patient data validation of the network-identified drugs using a COVID-19 registry. Institutional review board—approved registry COVID-19 registry data was used, including 18,118 individuals (1,675 positive) tested during Mar. 8 to Apr. 16, 2020 from the Cleveland Clinic Health System in Ohio and Florida. Data included COVID-19 test results, baseline demographic information, medications, and all recorded disease conditions and others. A series of retrospective case-control studies were conducted with an active comparator new user design to test the drug-outcome relationships for COVID-19. Data were extracted from electronic health records (EPIC Systems) and were manually checked by a study team trained on uniform sources for the study variables. All patient data was collected and managed using REDCap electronic data capture tools. The exposures of drugs (including carvedilol and melatonin) were used as recorded in the medication list in the electronic medical records at the time of testing for SARS-CoV-2. A positive laboratory test result for COVID-19 was used as the primary outcome. A propensity score (PS) was calculated for melatonin and carvedilol intake by multivariable logistic regression models with two sets of covariates: (1) PS-matched model1, age, gender, race and smoking; (2) PS-matched model2, age, gender, race, smoking, coronary artery disease, diabetes, hypertension and COPD. The nearest-neighbor algorithm was used to match the melatonin and carvedilol subjects and references subjects on the basis of PS ratio of 1:4. Two odds ratios (OR) models were used to estimate the COVID-19 positive risk between melatonin or carvedilol intaking groups and non-intaking groups: (1) OR model1, COVID-19 melatonin/carvedilol; (2) OR model2 (adjusted OR model), COVID-19˜melatonin/carvedilol+age, gender, race, smoking, coronary artery disease, diabetes, hypertension and COPD. All analyses were conducted by matchit package in the R v3.6.3 platform.

Statistical analysis and network visualization. Statistical tests were performed with the Python package SciPy v1.3.0. P<0.05 was considered statistically significant throughout this study. Networks were visualized using Gephi 0.9.2.

### REFERENCES

1. P. K. Bhatraju, B. J. Ghassemieh, M. Nichols, R. Kim, K. R. Jerome, A. K. Nalla, A. L. Greninger, S. Pipavath, M. M. Wurfel, L. Evans, P. A. Kritek, T. E. West, A. Luks, A. Gerbino, C. R. Dale, J. D. Goldman, S. O'Mahony, C. Mikacenic, Covid-19 in Critically Ill Patients in the Seattle Region—Case Series. The New England journal of medicine, (2020).
2. W. J. Guan, Z. Y. Ni, Y. Hu, W. H. Liang, C. Q. Ou, J. X. He, L. Liu, H. Shan, C. L. Lei, D. S. C. Hui, B. Du, L. J. Li, G. Zeng, K. Y. Yuen, R. C. Chen, C. L. Tang, T. Wang, P. Y. Chen, J. Xiang, S. Y. Li, J. L. Wang, Z. J. Liang, Y. X. Peng, L. Wei, Y. Liu, Y. H. Hu, P. Peng, J. M. Wang, J. Y. Liu, Z. Chen, G. Li, Z. J. Zheng, S. Q. Qiu, J. Luo, C. J. Ye, S. Y. Zhu, N. S. Zhong, Clinical Characteristics of Coronavirus Disease 2019 in China. The New England journal of medicine, (2020).
3. D. Wang, B. Hu, C. Hu, F. Zhu, X. Liu, J. Zhang, B. Wang, H. Xiang, Z. Cheng, Y. Xiong, Y. Zhao, Y. Li, X. Wang, Z. Peng, Clinical Characteristics of 138 Hospitalized Patients With 2019 Novel Coronavirus-Infected Pneumonia in Wuhan, China. Jama, (2020).
4. X. Yang, Y. Yu, J. Xu, H. Shu, J. Xia, H. Liu, Y. Wu, L. Zhang, Z. Yu, M. Fang, T. Yu, Y. Wang, S. Pan, X. Zou, S. Yuan, Y. Shang, Clinical course and outcomes of critically ill patients with SARS-CoV-2 pneumonia in Wuhan, China: a single-centered, retrospective, observational study. The Lancet. Respiratory medicine, (2020).
5. F. Zhou, T. Yu, R. Du, G. Fan, Y. Liu, Z. Liu, J. Xiang, Y. Wang, B. Song, X. Gu, L. Guan, Y. Wei, H. Li, X. Wu, J. Xu, S. Tu, Y. Zhang, H. Chen, B. Cao, Clinical course and risk factors for mortality of adult inpatients with COVID-19 in Wuhan, China: a retrospective cohort study. Lancet (London, England) 395, 1054-1062 (2020).
6. N. Chen, M. Zhou, X. Dong, J. Qu, F. Gong, Y. Han, Y. Qiu, J. Wang, Y. Liu, Y. Wei, J. Xia, T. Yu, X. Zhang, L. Zhang, Epidemiological and clinical characteristics of 99 cases of 2019 novel coronavirus pneumonia in Wuhan, China: a descriptive study. Lancet (London, England) 395, 507-513 (2020).
7. P. Zhou, X. L. Yang, X. G. Wang, B. Hu, L. Zhang, W. Zhang, H. R. Si, Y. Zhu, B. Li, C. L. Huang, H. D. Chen, J. Chen, Y. Luo, H. Guo, R. D. Jiang, M. Q. Liu, Y. Chen, X. R. Shen, X. Wang, X. S. Zheng, K. Zhao, Q. J. Chen, F. Deng, L. L. Liu, B. Yan, F. X. Zhan, Y. Y. Wang, G. F. Xiao, Z. L. Shi, A pneumonia outbreak associated with a new coronavirus of probable bat origin. Nature 579, 270-273 (2020).
8. N. Zhu, D. Zhang, W. Wang, X. Li, B. Yang, J. Song, X. Zhao, B. Huang, W. Shi, R. Lu, P. Niu, F. Zhan, X. Ma, D. Wang, W. Xu, G. Wu, G. F. Gao, W. Tan, I. China Novel Coronavirus, T. Research, A Novel Coronavirus from Patients with Pneumonia in China, 2019. The New England journal of medicine 382, 727-733 (2020).
9. M. Hoffmann, H. Kleine-Weber, S. Schroeder, N. Krüger, T. Herrler, S. Erichsen, T. S. Schiergens, G. Herrler, N.-H. Wu, A. Nitsche, M. A. Muller, C. Drosten, S. Pöhlmann, SARS-CoV-2 Cell Entry Depends on ACE2 and TMPRSS2 and Is Blocked by a Clinically Proven Protease Inhibitor. Cell 181, 271-280.e278 (2020).
10. M. Hoffmann, H. Kleine-Weber, S. Pohlmann, A Multibasic Cleavage Site in the Spike Protein of SARS-CoV-2 Is Essential for Infection of Human Lung Cells. Molecular Cell, (2020).
11. C. G. K. Ziegler, S. J. Allon, S. K. Nyquist, I. M. Mbano, V. N. Miao, C. N. Tzouanas, Y. Cao, A. S. Yousif, J. Bals, B. M. Hauser, J. Feldman, C. Muus, M. H. Wadsworth, S. W. Kazer, T. K. Hughes, B. Doran, G. J. Gaffer, M. Vukovic, F. Taliaferro, B. E. Mead, Z. Guo, J. P. Wang, D. Gras, M. Plaisant, M. Ansari, I. Angelidis, H. Adler, J. M. S. Sucre, C. J. Taylor, B. Lin, A. Waghray, V. Mitsialis, D. F. Dwyer, K. M. Buchheit, J. A. Boyce, N. A. Barrett, T. M. Laidlaw, S. L. Carroll, L. Colonna, V. Tkachev, C. W. Peterson, A. Yu, H. B. Zheng, H. P. Gideon, C. G. Winchell, P. L. Lin, C. D. Bingle, S. B. Snapper, J. A. Kropski, F. J. Theis, H. B. Schiller, L.-E. Zaragosi, P. Barbry, A. Leslie, H.-P. Kiem, J. L. Flynn, S. M. Fortune, B. Berger, R. W. Finberg, L. S. Kean, M. Garber, A. G. Schmidt, D. Lingwood, A. K. Shalek, J. Ordovas-Montanes, SARS-CoV-2 receptor ACE2 is an interferon-stimulated gene in human airway epithelial cells and is detected in specific cell subsets across tissues. Cell, (2020).
12. W. Sungnak, N. Huang, C. Bécavin, M. Berg, R. Queen, M. Litvinukova, C. Talavera-López, H. Maatz, D. Reichart, F. Sampaziotis, K. B. Worlock, M. Yoshida, J. L. Barnes, N. E. Banovich, P. Barbry, A. Brazma, J. Collin, T. J. Desai, T. E. Duong, O. Eickelberg, C. Falk, M. Farzan, I. Glass, R. K. Gupta, M. Haniffa, P. Horvath, N. Hubner, D. Hung, N. Kaminski, M. Krasnow, J. A. Kropski, M. Kuhnemund, M. Lako, H. Lee, S. Leroy, S. Linnarson, J. Lundeberg, K. B. Meyer, Z. Miao, A. V. Misharin, M. C. Nawijn, M. Z. Nikolic, M. Noseda, J. Ordovas-Montanes, G. Y. Oudit, D. Pe'er, J. Powell, S. Quake, J. Rajagopal, P. R. Tata, E. L. Rawlins, A. Regev, P. A. Reyfman, 0. Rozenblatt-Rosen, K. Saeb-Parsy, C. Samakovlis, H. B. Schiller, J. L. Schultze, M. A. Seibold, C. E. Seidman, J. G. Seidman, A. K. Shalek, D. Shepherd, J. Spence, A. Spira, X. Sun, S. A. Teichmann, F. J. Theis, A. M. Tsankov, L. Vallier, M. van den Berge, J. Whitsett, R. Xavier, Y. Xu, L.-E. Zaragosi, D. Zerti, H. Zhang, K. Zhang, M. Rojas, F. Figueiredo, H. C. A. L. B. Network, SARS-CoV-2 entry factors are highly expressed in nasal epithelial cells together with innate immune genes. Nature Medicine, (2020).
13. S. Lukassen, R. L. Chua, T. Trefzer, N. C. Kahn, M. A. Schneider, T. Muley, H. Winter, M. Meister, C. Veith, A. W. Boots, B. P. Hennig, M. Kreuter, C. Conrad, R. Eils, SARS-CoV-2 receptor ACE2 and TMPRSS2 are primarily expressed in bronchial transient secretory cells. EMBO J, e105114 (2020).
14. H. Zhang, Z. Kang, H. Gong, D. Xu, J. Wang, Z. Li, Z. Li, X. Cui, J. Xiao, J. Zhan, T. Meng, W. Zhou, J. Liu, H. Xu, Digestive system is a potential route of COVID-19: an analysis of single-cell coexpression pattern of key proteins in viral entry process. Gut, gutjnl-2020-320953 (2020).
15. S. Yang, C. Fu, X. Lian, X. Dong, Z. Zhang, Understanding Human-Virus Protein-Protein Interactions Using a Human Protein Complex-Based Analysis Framework. mSystems 4, (2019).
16. C. Liu, Y. Ma, J. Zhao, R. Nussinov, Y. Zhang, F. Cheng, Z. Zhang, Computational network biology: Data, models, and applications. DOI: 10.1016/j.physrep.2019.12.004. Physics Reports, (2020).
17. A. Zumla, J. F. Chan, E. I. Azhar, D. S. Hui, K. Y. Yuen, Coronaviruses - drug discovery and therapeutic options. Nat Rev Drug Discov 15, 327-347 (2016).
18. D. Blanco-Melo, B. E. Nilsson-Payant, W.-C. Liu, R. Moller, M. Panis, D. Sachs, R. A. Albrecht, B. R. tenOever, SARS-CoV-2 launches a unique transcriptional signature from in vitro, ex vivo, and in vivo systems. bioRxiv, 2020.2003.2024.004655 (2020).
19. D. Bojkova, K. Klann, B. Koch, M. Widera, D. Krause, S. Ciesek, J. Cinatl, C. Munch, Proteomics of SARS-CoV-2-infected host cells reveals therapy targets. Nature, (2020).
20. D. E. Gordon, G. M. Jang, M. Bouhaddou, J. Xu, K. Obernier, K. M. White, M. J. O'Meara, V. V. Rezelj, J. Z. Guo, D. L. Swaney, T. A. Tummino, R. Huettenhain, R. M. Kaake, A. L. Richards, B. Tutuncuoglu, H. Foussard, J. Batra, K. Haas, M. Modak, M. Kim, P. Haas, B. J. Polacco, H. Braberg, J. M. Fabius, M. Eckhardt, M. Soucheray, M. J. Bennett, M. Cakir, M. J. McGregor, Q. Li, B. Meyer, F. Roesch, T. Vallet, A. Mac Kain, L. Miorin, E. Moreno, Z. Z. C. Naing, Y. Zhou, S. Peng, Y. Shi, Z. Zhang, W. Shen, I. T. Kirby, J. E. Melnyk, J. S. Chorba, K. Lou, S. A. Dai, I. Barrio-Hernandez, D. Memon, C. Hernandez-Armenta, J. Lyu, C. J. P. Mathy, T. Perica, K. B. Pilla, S. J. Ganesan, D. J. Saltzberg, R. Rakesh, X. Liu, S. B. Rosenthal, L. Calviello, S. Venkataramanan, J. Liboy-Lugo, Y. Lin, X. P. Huang, Y. Liu, S. A. Wankowicz, M. Bohn, M. Safari, F. S. Ugur, C. Koh, N. S. Savar, Q. D. Tran, D. Shengjuler, S. J. Fletcher, M. C. O'Neal, Y. Cai, J. C. J. Chang, D. J. Broadhurst, S. Klippsten, P. P. Sharp, N. A. Wenzell, D. Kuzuoglu, H. Y. Wang, R. Trenker, J. M. Young, D. A. Cavero, J. Hiatt, T. L. Roth, U. Rathore, A. Subramanian, J. Noack, M. Hubert, R. M. Stroud, A. D. Frankel, O. S. Rosenberg, K. A. Verba, D. A. Agard, M. Ott, M. Emerman, N. Jura, M. von Zastrow, E. Verdin, A. Ashworth, O. Schwartz, C. d'Enfert, S. Mukherjee, M. Jacobson, H. S. Malik, D. G. Fujimori, T. Ideker, C. S. Craik, S. N. Floor, J. S. Fraser, J. D. Gross, A. Sali, B. L. Roth, D. Ruggero, J. Taunton, T. Kortemme, P. Beltrao, M. Vignuzzi, A. Garcia-Sastre, K. M. Shokat, B. K. Shoichet, N. J. Krogan, A SARS-CoV-2 protein interaction map reveals targets for drug repurposing. Nature, doi: 10.1038/s41586-020-2286-9 (2020).
21. F. Cheng, In Silico Oncology Drug Repositioning and Polypharmacology. Methods Mol Biol 1878, 243-261 (2019).
22. F. Cheng, H. Hong, S. Yang, Y. Wei, Individualized network-based drug repositioning infrastructure for precision oncology in the panomics era. Brief Bioinform 18, 682-697 (2017).
23. F. Cheng, J. L. Murray, D. H. Rubin, Drug Repurposing: New Treatments for Zika Virus Infection? Trends Mol Med 22, 919-921 (2016).
24. F. Cheng, R. J. Desai, D. E. Handy, R. Wang, S. Schneeweiss, A. L. Barabasi, J. Loscalzo, Network-based approach to prediction and population-based validation of in silico drug repurposing. Nat Commun 9, 2691 (2018).
25. F. Cheng, J. L. Murray, J. Zhao, J. Sheng, Z. Zhao, D. H. Rubin, Systems Biology-Based Investigation of Cellular Antiviral Drug Targets Identified by Gene-Trap Insertional Mutagenesis. PLoS Comput Biol 12, e1005074 (2016).
26. B. Cao, Y. Wang, D. Wen, W. Liu, J. Wang, G. Fan, L. Ruan, B. Song, Y. Cai, M. Wei, X. Li, J. Xia, N. Chen, J. Xiang, T. Yu, T. Bai, X. Xie, L. Zhang, C. Li, Y. Yuan, H. Chen, H. Li, H. Huang, S. Tu, F. Gong, Y. Liu, Y. Wei, C. Dong, F. Zhou, X. Gu, J. Xu, Z. Liu, Y. Zhang, H. Li, L. Shang, K. Wang, K. Li, X. Zhou, X. Dong, Z. Qu, S. Lu, X. Hu, S. Ruan, S. Luo, J. Wu, L. Peng, F. Cheng, L. Pan, J. Zou, C. Jia, J. Wang, X. Liu, S. Wang, X. Wu, Q. Ge, J. He, H. Zhan, F. Qiu, L. Guo, C. Huang, T. Jaki, F. G. Hayden, P. W. Horby, D. Zhang, C. Wang, A Trial of Lopinavir-Ritonavir in Adults Hospitalized with Severe Covid-19. New England Journal of Medicine, (2020).
27. L. D. CHEN Jun, LIU Li, LIU Ping, X U Qingnian, XIA Lu, LING Yun, HUANG Dan, SONG Shuli, ZHANG Dandan, QIAN Zhiping, L I Tao, SHEN Yinzhong, L U Hongzhou, A pilot study of hydroxychloroquine in treatment of patients with common coronavirus disease-19 (COVID-19). J Zhejiang Univ (Med Sci) 49, 0-0 (2020).
28. Y. Zhou, Y. Hou, J. Shen, Y. Huang, W. Martin, F. Cheng, Network-based drug repurposing for novel coronavirus 2019-nCoV/SARS-CoV-2. Cell Discov 6, 14 (2020).
29. F. Cheng, W. Lu, C. Liu, J. Fang, Y. Hou, D. E. Handy, R. Wang, Y. Zhao, Y. Yang, J. Huang, D. E. Hill, M. Vidal, C. Eng, J. Loscalzo, A genome-wide positioning systems network algorithm for in silico drug repurposing. Nat Commun 10, 3476 (2019).
30. F. Cheng, I. A. Kovacs, A. L. Barabasi, Network-based prediction of drug combinations. Nat Commun 10, 1197 (2019).
31. D. M. Gysi, I. D. Valle, M. Zitnik, A. Ameli, X. Gan, O. Varol, H. Sanchez, R. M. Baron, D. Ghiassian, J. Loscalzo, A.-L. Barabási, Network Medicine Framework for Identifying Drug Repurposing Opportunities for COVID-19. arXiv, 2004.07229 (2020).
32. G. Li, X. He, L. Zhang, Q. Ran, J. Wang, A. Xiong, D. Wu, F. Chen, J. Sun, C. Chang, Assessing ACE2 expression patterns in lung tissues in the pathogenesis of COVID-19. J Autoimmun, 102463 (2020).
33. H. Shimokawa, S. Sunamura, K. Satoh, RhoA/Rho-Kinase in the Cardiovascular System. Circ Res 118, 352-366 (2016).
34. K. Zaoui, Z. Boudhraa, P. Khalife, E. Carmona, D. Provencher, A. M. Mes-Masson, Ran promotes membrane targeting and stabilization of RhoA to orchestrate ovarian cancer cell invasion. Nat Commun 10, 2666 (2019).
35. S. A. Forbes, N. Bindal, S. Bamford, C. Cole, C. Y. Kok, D. Beare, M. Jia, R. Shepherd, K. Leung, A. Menzies, J. W. Teague, P. J. Campbell, M. R. Stratton, P. A. Futreal, COSMIC: mining complete cancer genomes in the Catalogue of Somatic Mutations in Cancer. Nucleic Acids Res 39, D945-950 (2011).
36. C. Liu, J. Zhao, W. Lu, Y. Dai, J. Hockings, Y. Zhou, R. Nussinov, C. Eng, F. Cheng, Individualized genetic network analysis reveals new therapeutic vulnerabilities in 6,700 cancer genomes. PLoS Comput Biol 16, e1007701 (2020).
37. P. D. Stenson, E. V. Ball, M. Mort, A. D. Phillips, J. A. Shiel, N. S. Thomas, S. Abeysinghe, M. Krawczak, D. N. Cooper, Human Gene Mutation Database (HGMD): 2003 update. Hum Mutat 21, 577-581 (2003).
38. K. J. Clerkin, J. A. Fried, J. Raikhelkar, G. Sayer, J. M. Griffin, A. Masoumi, S. S. Jain, D. Burkhoff, D. Kumaraiah, L. Rabbani, A. Schwartz, N. Uriel, Coronavirus Disease 2019 (COVID-19) and Cardiovascular Disease. Circulation, (2020).
39. E. Baldi, G. M. Sechi, C. Mare, F. Canevari, A. Brancaglione, R. Primi, C. Klersy, A. Palo, E. Contri, V. Ronchi, G. Beretta, F. Reali, P. Parogni, F. Facchin, D. Bua, U. Rizzi, D. Bussi, S. Ruggeri, L. Oltrona Visconti, S. Savastano, Out-of-Hospital Cardiac Arrest during the Covid-19 Outbreak in Italy. New England Journal of Medicine, (2020).
40. M. F. Beers, S. Mulugeta, The biology of the ABCA3 lipid transporter in lung health and disease. Cell Tissue Res 367, 481-493 (2017).
41. L. M. Nogee, Abnormal expression of surfactant protein C and lung disease. Am J Respir Cell Mol Biol 26, 641-644 (2002).
42. M. Z. Tay, C. M. Poh, L. Rénia, P. A. MacAry, L. F. P. Ng, The trinity of COVID-19: immunity, inflammation and intervention. Nature Reviews Immunology, (2020).
43. D. Swaroopa, K. Bhaskar, T. Mahathi, S. Katkam, Y. S. Raju, N. Chandra, V. K. Kutala, Association of serum interleukin-6, interleukin-8, and Acute Physiology and Chronic Health Evaluation II score with clinical outcome in patients with acute respiratory distress syndrome. Indian J Crit Care Med 20, 518-525 (2016).
44. T. Hou, D. Huang, R. Zeng, Z. Ye, Y. Zhang, Accuracy of serum interleukin (IL)-6 in sepsis diagnosis: a systematic review and meta-analysis. Int J Clin Exp Med 8, 15238-15245 (2015).
45. J. Song, D. W. Park, S. Moon, H.-J. Cho, J. H. Park, H. Seok, W. S. Choi, Diagnostic and prognostic value of interleukin-6, pentraxin 3, and procalcitonin levels among sepsis and septic shock patients: a prospective controlled study according to the Sepsis-3 definitions. BMC Infectious Diseases 19, 968 (2019).
46. X. Xu, M. Han, T. Li, W. Sun, D. Wang, B. Fu, Y. Zhou, X. Zheng, Y. Yang, X. Li, X. Zhang, A. Pan, H. Wei, Effective treatment of severe COVID-19 patients with tocilizumab. Proceedings of the National Academy of Sciences, 202005615 (2020).
47. Q. Wang, P. Fang, R. He, M. Li, H. Yu, L. Zhou, Y. Yi, F. Wang, Y. Rong, Y. Zhang, A. Chen, N. Peng, Y. Lin, M. Lu, Y. Zhu, G. Peng, L. Rao, S. Liu, O-GlcNAc transferase promotes influenza A virus-induced cytokine storm by targeting interferon regulatory factor-5. Science Advances 6, eaaz7086 (2020).
48. S. A. Comhair, J. McDunn, C. Bennett, J. Fettig, S. C. Erzurum, S. C. Kalhan, Metabolomic Endotype of Asthma. J Immunol 195, 643-650 (2015).
49. F. Holguin, H. Grasemann, S. Sharma, D. Winnica, K. Wasil, V. Smith, M. H. Cruse, N. Perez, E. Coleman, T. J. Scialla, L. G. Que, L-Citrulline increases nitric oxide and improves control in obese asthmatics. JCI Insight 4, (2019).
50. B. Shen, X. Yi, Y. Sun, X. Bi, J. Du, C. Zhang, S. Quan, F. Zhang, R. Sun, L. Qian, W. Ge, W. Liu, S. Liang, H. Chen, Y. Zhang, J. Li, J. Xu, Z. He, B. Chen, J. Wang, H. Yan, Y. Zheng, D. Wang, J. Zhu, Z. Kong, Z. Kang, X. Liang, X. Ding, G. Ruan, N. Xiang, X. Cai, H. Gao, L. Li, S. Li, Q. Xiao, T. Lu, Y. J. Zhu, H. Liu, H. Chen, T. Guo, Proteomic and Metabolomic Characterization of COVID-19 Patient Sera. medRxiv, 2020.2004.2007.20054585 (2020).
51. B. Samuelsson, Arachidonic acid metabolism: role in inflammation. Z Rheumatol 50 Suppl 1, 3-6 (1991).
52. C. Calabrese, M. Triggiani, G. Marone, G. Mazzarella, Arachidonic acid metabolism in inflammatory cells of patients with bronchial asthma. Allergy 55 Suppl 61, 27-30 (2000).
53. A. Berger, What are leukotrienes and how do they work in asthma? BMJ 319, 90 (1999).
54. B. D. Modena, J. R. Tedrow, J. Milosevic, E. R. Bleecker, D. A. Meyers, W. Wu, Z. Bar-Joseph, S. C. Erzurum, B. M. Gaston, W. W. Busse, N. N. Jarjour, N. Kaminski, S. E. Wenzel, Gene expression in relation to exhaled nitric oxide identifies novel asthma phenotypes with unique biomolecular pathways. Am JRespir Crit Care Med 190, 1363-1372 (2014).
55. N. Weathington, M. E. O'Brien, J. Radder, T. C. Whisenant, E. R. Bleecker, W. W. Busse, S. C. Erzurum, B. Gaston, A. T. Hastie, N. N. Jarjour, D. A. Meyers, J. Milosevic, W. C. Moore, J. R. Tedrow, J. B. Trudeau, H. P. Wong, W. Wu, N. Kaminski, S. E. Wenzel, B. D. Modena, BAL Cell Gene Expression in Severe Asthma Reveals Mechanisms of Severe Disease and Influences of Medications. Am JRespir Crit Care Med 200, 837-856 (2019).
56. L. Balaci, M. C. Spada, N. 011a, G. Sole, L. Loddo, F. Anedda, S. Naitza, M. A. Zuncheddu, A. Maschio, D. Altea, M. Uda, S. Pilia, S. Sanna, M. Masala, L. Crisponi, M. Fattori, M. Devoto, S. Doratiotto, S. Rassu, S. Mereu, E. Giva, N. G. Cadeddu, R. Atzeni, U. Pelosi, A. Corrias, R. Perra, P. L. Torrazza, P. Pirina, F. Ginesu, S. Marcias, M. G. Schintu, G. S. D. Giacco, P. E. Manconi, G. Malerba, A. Bisognin, E. Trabetti, A. Boner, L. Pescollderungg, P. F. Pignatti, D. Schlessinger, A. Cao, G. Pilia, IRAK-M Is Involved in the Pathogenesis of Early-Onset Persistent Asthma. The American Journal of Human Genetics 80, 1103-1114 (2007).
57. A. C. Z. de Paiva, F. A. d. L. Marson, J. D. Ribeiro, C. S. Bertuzzo, Asthma: Gln27Glu and Arg16Gly polymorphisms of the beta2-adrenergic receptor gene as risk factors. Allergy, Asthma & Clinical Immunology 10, 8 (2014).
58. M. M. Lamers, J. Beumer, J. van der Vaart, K. Knoops, J. Puschhof, T. I. Breugem, R. B. G. Ravelli, J. Paul van Schayck, A. Z. Mykytyn, H. Q. Duimel, E. van Donselaar, S. Riesebosch, H. J. H. Kuijpers, D. Schippers, W. J. van de Wetering, M. de Graaf, M. Koopmans, E. Cuppen, P. J. Peters, B. L. Haagmans, H. Clevers, SARS-CoV-2 productively infects human gut enterocytes. Science, eabc1669 (2020).
59. F. D'Amico, D. C. Baumgart, S. Danese, L. Peyrin-Biroulet, Diarrhea During COVID-19 Infection: Pathogenesis, Epidemiology, Prevention, and Management. Clin Gastroenterol Hepatol, (2020).
60. J. C. Martin, C. Chang, G. Boschetti, R. Ungaro, M. Giri, J. A. Grout, K. Gettler, L. S. Chuang, S. Nayar, A. J. Greenstein, M. Dubinsky, L. Walker, A. Leader, J. S. Fine, C. E. Whitehurst, M. L. Mbow, S. Kugathasan, L. A. Denson, J. S. Hyams, J. R. Friedman, P. T. Desai, H. M. Ko, I. Laface, G. Akturk, E. E. Schadt, H. Salmon, S. Gnjatic, A. H. Rahman, M. Merad, J. H. Cho, E. Kenigsberg, Single-Cell Analysis of Crohn's Disease Lesions Identifies a Pathogenic Cellular Module Associated with Resistance to Anti-TNF Therapy. Cell 178, 1493-1508 e1420 (2019).
61. W. Zhang, K. Y. Hui, A. Gusev, N. Warner, S. M. Ng, J. Ferguson, M. Choi, A. Burberry, C. Abraham, L. Mayer, R. J. Desnick, C. J. Cardinale, H. Hakonarson, M. Waterman, Y. Chowers, A. Karban, S. R. Brant, M. S. Silverberg, P. K. Gregersen, S. Katz, R. P. Lifton, H. Zhao, G. Nunez, I. Pe'er, I. Peter, J. H. Cho, Extended haplotype association study in Crohn's disease identifies a novel, Ashkenazi Jewish-specific missense mutation in the NF-kappaB pathway gene, HEATR3. Genes Immun 14, 310-316 (2013).
62. A. M. Muise, T. Walters, W. Xu, G. Shen—Tu, C. H. Guo, R. Fattouh, G. Y. Lam, V. M. Wolters, J. Bennitz, J. van Limbergen, P. Renbaum, Y. Kasirer, B. Y. Ngan, D. Turner, L. A. Denson, P. M. Sherman, R. H. Duerr, J. Cho, C. W. Lees, J. Satsangi, D. C. Wilson, A. D. Paterson, A. M. Griffiths, M. Glogauer, M. S. Silverberg, J. H. Brumell, Single Nucleotide Polymorphisms That Increase Expression of the Guanosine Triphosphatase RAC1 Are Associated With Ulcerative Colitis. Gastroenterology 141, 633-641 (2011).
63. M. L. Seinen, G. P. van Nieuw Amerongen, N. K. H. de Boer, C. J. J. Mulder, J. van Bezu, A. A. van Bodegraven, Racl as a Potential Pharmacodynamic Biomarker for Thiopurine Therapy in Inflammatory Bowel Disease. Therapeutic Drug Monitoring 38, 621-627 (2016).
64. B. I. Korelitz, Expert opinion: experience with 6-mercaptopurine in the treatment of inflammatory bowel disease. World J Gastroenterol 19, 2979-2984 (2013).
65. C. M. Perry, L. J. Scott, Cefdinir: A Review of its Use in the Management of Mild-to-Moderate Bacterial Infections. Drugs 64, 1433-1464 (2004).
66. L. M. Johansen, J. M. Brannan, S. E. Delos, C. J. Shoemaker, A. Stossel, C. Lear, B. G. Hoffstrom, L. E. Dewald, K. L. Schornberg, C. Scully, J. Lehar, L. E. Hensley, J. M. White, G. G. Olinger, FDA-approved selective estrogen receptor modulators inhibit Ebola virus infection. Sci Transl Med 5, 190ra179 (2013).
67. Y. Cong, B. J. Hart, R. Gross, H. Zhou, M. Frieman, L. Bollinger, J. Wada, L. E. Hensley, P. B. Jahrling, J. Dyall, M. R. Holbrook, MERS-CoV pathogenesis and antiviral efficacy of licensed drugs in human monocyte-derived antigen-presenting cells. PLoS One 13, e0194868 (2018).
68. J. Dyall, C. M. Coleman, B. J. Hart, T. Venkataraman, M. R. Holbrook, J. Kindrachuk, R. F. Johnson, G. G. Olinger, Jr., P. B. Jahrling, M. Laidlaw, L. M. Johansen, C. M. Lear-Rooney, P. J. Glass, L. E. Hensley, M. B. Frieman, Repurposing of clinically developed drugs for treatment of Middle East respiratory syndrome coronavirus infection. Antimicrob Agents Chemother 58, 4885-4893 (2014).
69. S. Jeon, M. Ko, J. Lee, I. Choi, Y. Byun, S. Park, D. Shum, K. S., Identification of antiviral drug candidates against SARS-CoV-2 from FDA-approved drugs. bioRxiv, (2020).
70. X. J. Wang, W. Hu, T. Y. Zhang, Y. Y. Mao, N. N. Liu, S. Q. Wang, Irbesartan, an FDA approved drug for hypertension and diabetic nephropathy, is a potent inhibitor for hepatitis B virus entry by disturbing Na(+)-dependent taurocholate cotransporting polypeptide activity. Antiviral Res 120, 140-146 (2015).
71. P. Zhang, L. Zhu, J. Cai, F. Lei, J.-J. Qin, J. Xie, Y.-M. Liu, Y.-C. Zhao, X. Huang, L. Lin, M. Xia, M.-M. Chen, X. Cheng, X. Zhang, D. Guo, Y. Peng, Y.-X. Ji, J. Chen, Z.-G. She, Y. Wang, Q. Xu, R. Tan, H. Wang, J. Lin, P. Luo, S. Fu, H. Cai, P. Ye, B. Xiao, W. Mao, L. Liu, Y. Yan, M. Liu, M. Chen, X.-J. Zhang, X. Wang, R. M. Touyz, J. Xia, B.-H. Zhang, X. Huang, Y. Yuan, L. Rohit, P. P. Liu, H. Li, Association of Inpatient Use of Angiotensin Converting Enzyme Inhibitors and Angiotensin II Receptor Blockers with Mortality Among Patients With Hypertension Hospitalized With COVID-19. Circulation Research 0.
72. V. G. Puelles, M. Lutgehetmann, M. T. Lindenmeyer, J. P. Sperhake, M. N. Wong, L. Allweiss, S. Chilla, A. Heinemann, N. Wanner, S. Liu, F. Braun, S. Lu, S. Pfefferle, A. S. Schroder, C. Edler, O. Gross, M. Glatzel, D. Wichmann, T. Wiech, S. Kluge, K. Pueschel, M. Aepfelbacher, T. B. Huber, Multiorgan and Renal Tropism of SARS-CoV-2. The New England journal of medicine, (2020).
73. F. Xiao, M. Tang, X. Zheng, Y. Liu, X. Li, H. Shan, Evidence for Gastrointestinal Infection of SARS-CoV-2. Gastroenterology 158, 1831-1833 e1833 (2020).
74. K. Nakashima, T. Hirota, K. Obara, M. Shimizu, A. Jodo, M. Kameda, S. Doi, K. Fujita, T. Shirakawa, T. Enomoto, F. Kishi, S. Yoshihara, K. Matsumoto, H. Saito, Y. Suzuki, Y. Nakamura, M. Tamari, An association study of asthma and related phenotypes with polymorphisms in negative regulator molecules of the TLR signaling pathway. Journal of Human Genetics 51, 284-291 (2006).
75. M. Pino-Yanes, I. Sanchez-Machin, J. Cumplido, J. Figueroa, M. J. Torres-Galvan, R. Gonzalez, A. Corrales, O. Acosta-Fernandez, J. C. Garcia-Robaina, T. Carrillo, A. Sanchez-Palacios, J. Villar, M. Hernandez, C. Flores, IL-1 receptor-associated kinase 3 gene (IRAK3) variants associate with asthma in a replication study in the Spanish population. J Allergy Clin Immunol 129, 573-575, 575 e571-510 (2012).
76. E. Reihsaus, M. Innis, N. Maclntyre, S. B. Liggett, Mutations in the gene encoding for the beta 2-adrenergic receptor in normal and asthmatic subjects. Am J Respir Cell Mol Biol 8, 334-339 (1993).
77. S. Ali, A. F. Hirschfeld, M. L. Mayer, E. S. Fortuno, N. Corbett, M. Kaplan, S. Wang, J. Schneiderman, C. D. Fjell, J. Yan, L. Akhabir, F. Aminuddin, N. Man, T. Lacaze-Masmonteil, R. G. Hegele, A. Becker, M. Chan-Yeung, R. E. W. Hancock, T. R. Kollmann, D. Daley, A. J. Sandford, P. M. Lavoie, S. E. Turvey, Functional Genetic Variation in NFKBIA and Susceptibility to Childhood Asthma, Bronchiolitis, and Bronchopulmonary Dysplasia. The Journal of Immunology 190, 3949-3958 (2013).
78. T. Akaike, H. Maeda, Nitric oxide and virus infection. Immunology 101, 300-308 (2000).
79. R. Verity, L. C. Okell, I. Dorigatti, P. Winskill, C. Whittaker, N. Imai, G. Cuomo-Dannenburg, H. Thompson, P. G. T. Walker, H. Fu, A. Dighe, J. T. Griffin, M. Baguelin, S. Bhatia, A. Boonyasiri, A. Cori, Z. Cucunuba, R. FitzJohn, K. Gaythorpe, W. Green, A. Hamlet, W. Hinsley, D. Laydon, G. Nedjati-Gilani, S. Riley, S. van Elsland, E. Volz, H. Wang, Y. Wang, X. Xi, C. A. Donnelly, A. C. Ghani, N. M. Ferguson, Estimates of the severity of coronavirus disease 2019: a model-based analysis. Lancet Infect Dis, (2020).
80. D. Aw, A. B. Silva, D. B. Palmer, Immunosenescence: emerging challenges for an ageing population. Immunology 120, 435-446 (2007).
81. X. Wang, Y. Bian, R. Zhang, X. Liu, L. Ni, B. Ma, R. Zeng, Z. Zhao, X. Song, C. Liu, Melatonin alleviates cigarette smoke-induced endothelial cell pyroptosis through inhibiting ROS/NLRP3 axis. Biochem Biophys Res Commun 519, 402-408 (2019).
82. Y. Shang, S. P. Xu, Y. Wu, Y. X. Jiang, Z. Y. Wu, S. Y. Yuan, S. L. Yao, Melatonin reduces acute lung injury in endotoxemic rats. Chin Med J (Engl) 122, 1388-1393 (2009).
83. J. Zhang, L. Wang, W. Xie, S. Hu, H. Zhou, P. Zhu, H. Zhu, Melatonin attenuates ER stress and mitochondrial damage in septic cardiomyopathy: A new mechanism involving BAP31 upregulation and MAPK-ERK pathway. J Cell Physiol 235, 2847-2856 (2020).
84. A. Hamosh, A. F. Scott, J. S. Amberger, C. A. Bocchini, V. A. McKusick, Online Mendelian Inheritance in Man (OMIM), a knowledgebase of human genes and genetic disorders. Nucleic Acids Res 33, D514-517 (2005).
85. M. Zhang, C. Zhu, A. Jacomy, L. J. Lu, A. G. Jegga, The orphan disease networks. Am J Hum Genet 88, 755-766 (2011).
86. R. Kittler, L. Pelletier, A.-K. Heninger, M. Slabicki, M. Theis, L. Miroslaw, I. Poser, S. Lawo, H. Grabner, K. Kozak, J. Wagner, V. Surendranath, C. Richter, W. Bowen, A. L. Jackson, B. Habermann, A. A. Hyman, F. Buchholz, Genome-scale RNAi profiling of cell division in human tissue culture cells. Nature Cell Biology 9, 1401-1412 (2007).
87. K. Breuer, A. K. Foroushani, M. R. Laird, C. Chen, A. Sribnaia, R. Lo, G. L. Winsor, R. E. W. Hancock, F. S. L. Brinkman, D. J. Lynn, InnateDB: systems biology of innate immunity and beyond—recent updates and continuing curation. Nucleic Acids Res. 41, D1228-D1233 (2012).
88. M. V. Kuleshov, M. R. Jones, A. D. Rouillard, N. F. Fernandez, Q. Duan, Z. Wang, S. Koplev, S. L. Jenkins, K. M. Jagodnik, A. Lachmann, M. G. McDermott, C. D. Monteiro, G. W. Gundersen, A. Ma'ayan, Enrichr: a comprehensive gene set enrichment analysis web server 2016 update. Nucleic Acids Res. 44, W90-W97 (2016).
89. A. E. Hirsh, H. B. Fraser, D. P. Wall, Adjusting for selection on synonymous sites in estimates of evolutionary distance. Mol Biol Evol 22, 174-177 (2005).
90. A. Bezginov, G. W. Clark, R. L. Charlebois, V. U. Dar, E. R. Tillier, Coevolution reveals a network of human proteins originating with multicellularity. Mol Biol Evol 30, 332-346 (2013).
91. F. Cheng, P. Jia, Q. Wang, C.-C. Lin, W.-H. Li, Z. Zhao, Studying Tumorigenesis through Network Evolution and Somatic Mutational Perturbations in the Cancer Interactome. Molecular Biology and Evolution 31, 2156-2169 (2014).
92. N. Mantel, W. Haenszel, Statistical Aspects of the Analysis of Data From Retrospective Studies of Disease. JNCI: Journal of the National Cancer Institute 22, 719-748 (1959).
93. R. DerSimonian, N. Laird, Meta-analysis in clinical trials. Controlled Clinical Trials 7, 177-188 (1986).
94. X. Wan, W. Wang, J. Liu, T. Tong, Estimating the sample mean and standard deviation from the sample size, median, range and/or interquartile range. BMC Medical Research Methodology 14, 135 (2014).
95. T. Stijnen, T. H. Hamza, P. Özdemir, Random effects meta-analysis of event outcome in the framework of the generalized linear mixed model with applications in sparse data. Statistics in Medicine 29, 3046-3067 (2010).
96. N. R. Coordinators, Database resources of the National Center for Biotechnology Information. Nucleic Acids Res 44, D7-19 (2016).
97. I. N. Smith, S. Thacker, M. Seyfi, F. Cheng, C. Eng, Conformational Dynamics and Allosteric Regulation Landscapes of Germline PTEN Mutations Associated with Autism Compared to Those Associated with Cancer. Am J Hum Genet 104, 861-878 (2019).
98. A. Butler, P. Hoffman, P. Smibert, E. Papalexi, R. Satija, Integrating single-cell transcriptomic data across different conditions, technologies, and species. Nat Biotechnol 36, 411-420 (2018).
99. V. Law, C. Knox, Y. Djoumbou, T. Jewison, A. C. Guo, Y. Liu, A. Maciejewski, D. Arndt, M. Wilson, V. Neveu, A. Tang, G. Gabriel, C. Ly, S. Adamjee, Z. T. Dame, B. Han, Y. Zhou, D. S. Wishart, DrugBank 4.0: shedding new light on drug metabolism. Nucleic Acids Res 42, D1091-1097 (2014).
100. H. Yang, C. Qin, Y. H. Li, L. Tao, J. Zhou, C. Y. Yu, F. Xu, Z. Chen, F. Zhu, Y. Z. Chen, Therapeutic target database update 2016: enriched resource for bench to clinical drug target and targeted pathway information. Nucleic Acids Res 44, D1069-1074 (2016).
101. A. Gaulton, L. J. Bellis, A. P. Bento, J. Chambers, M. Davies, A. Hersey, Y. Light, S. McGlinchey, D. Michalovich, B. Al-Lazikani, J. P. Overington, ChEMBL: a large-scale bioactivity database for drug discovery. Nucleic Acids Res. 40, D1100-D1107 (2012).
102. T. Q. Liu, Y. M. Lin, X. Wen, R. N. Jorissen, M. K. Gilson, BindingDB: a web-accessible database of experimentally determined protein-ligand binding affinities. Nucleic Acids Res. 35, D198-D201 (2007).
103. A. J. Pawson, J. L. Sharman, H. E. Benson, E. Faccenda, S. P. H. Alexander, O. P. Buneman, A. P. Davenport, J. C. McGrath, J. A. Peters, C. Southan, M. Spedding, W. Y. Yu, A. J. Harmar, I. Nc, The IUPHAR/BPS Guide to PHARMACOLOGY: an expert-driven knowledgebase of drug targets and their ligands. Nucleic Acids Res. 42, D1098-D1106 (2014).
104. J. Lamb, E. D. Crawford, D. Peck, J. W. Modell, I. C. Blat, M. J. Wrobel, J. Lerner, J. P. Brunet, A. Subramanian, K. N. Ross, M. Reich, H. Hieronymus, G. Wei, S. A. Armstrong, S. J. Haggarty, P. A. Clemons, R. Wei, S. A. Carr, E. S. Lander, T. R. Golub, The Connectivity Map: using gene-expression signatures to connect small molecules, genes, and disease. Science 313, 1929-1935 (2006).
105. R. Reghunathan, M. Jayapal, L.-Y. Hsu, H.-H. Chng, D. Tai, B. P. Leung, A. J. Melendez, Expression profile of immune response genes in patients with Severe Acute Respiratory Syndrome. BMC Immunology 6, 2 (2005).
106. L. Josset, V. D. Menachery, L. E. Gralinski, S. Agnihothram, P. Soya, V. S. Carter, B. L. Yount, R. L. Graham, R. S. Baric, M. G. Katze, Cell host response to infection with novel human coronavirus EMC predicts potential antivirals and important differences with SARS coronavirus. mBio 4, e00165-00113 (2013).
107. S. Yuan, H. Chu, J. F.-W. Chan, Z.-W. Ye, L. Wen, B. Yan, P.-M. Lai, K.-M. Tee, J. Huang, D. Chen, C. Li, X. Zhao, D. Yang, M. C. Chiu, C. Yip, V. K.-M. Poon, C. C.-S. Chan, K.-H. Sze, J. Zhou, I. H.-Y. Chan, K.-H. Kok, K. K.-W. To, R. Y.-T. Kao, J. Y.-N. Lau, D.-Y. Jin, S. Perlman, K.-Y. Yuen, SREBP-dependent lipidomic reprogramming as a broad-spectrum antiviral target. Nature Communications 10, 120 (2019).
108. Zeng X, Zhu S, Lu W, Liu Z, Huang J, Zhou Y, Fnag J, Huang Y, Guo H, Li L, Trapp B, Nussinov R, Eng C, Loscalzo J, Cheng F (2020) Target identification among known drugs by deep learning from heterogeneous networks. Chemical Science, 11, 1775-1797.
109. Huang Y, Fang J, Wang Z, Lu W, Wang Q, Hou Y, Jiang X, Reizes O, Lathia J, Nussinov R, Eng C, Cheng F (2019) A systems pharmacology approach uncovers wogonoside as a novel angiogenesis inhibitor of triple-negative breast cancer by targeting Hedgehog signaling, Cell Chemical Biology, 26: 1143-1158. PMCID: PMC6697584

All publications and patents mentioned in the present application are herein incorporated by reference. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A method of treating a viral infection in a subject comprising:
administering a therapeutically effective amount of an estrogen receptor modulator, or a pharmaceutically acceptable salt thereof, and a therapeutically effective amount of an anti-inflammatory agent, or a pharmaceutically acceptable salt thereof, to the subject,
wherein the subject is infected with a virus.

2. The method of claim 1, wherein the estrogen receptor modulator is selected from the group consisting of: tamoxifen, toremifene, clomifene, or pharmaceutically acceptable salts and combinations thereof.

3. The method of claim 1 or 2, wherein the estrogen receptor modulator comprises toremifene, or a pharmaceutically acceptable salt thereof.

4. The method of any one of claims 1-3, wherein the anti-inflammatory agent comprises melatonin.

5. The method of any one of claims 1-4, wherein the estrogen receptor modulator and anti-inflammatory agent, or pharmaceutically acceptable salts thereof, are administered simultaneously or sequentially in any order.

6. The method of any one of claims 1-5, wherein the estrogen receptor modulator and anti-inflammatory agent, or pharmaceutically acceptable salts thereof, are provided in individual pharmaceutical compositions.

7. The method of any one of claims 1-6, wherein the virus is a coronavirus.

8. The method of any one of claims 1-7, wherein the virus is selected from the group consisting of severe acute respiratory syndrome coronavirus-1 (SARS-CoV-1), severe acute respiratory syndrome coronavirus-2 (SARS-CoV-2), Middle East respiratory syndrome coronavirus (MERS-CoV), and Ebola virus.

9. The method of any one of claims 1-8, wherein the viral disease is human coronavirus disease 2019 (COVID-19).

10. A method for treating a coronavirus infection in a subject, comprising the steps of:
administering to the subject a therapeutically effective amount of an estrogen receptor modulator, or a pharmaceutically acceptable salt thereof, and a therapeutically effective amount of an anti-inflammatory agent, or a pharmaceutically acceptable salt thereof

11. The method of claim 10, wherein the coronavirus is severe acute respiratory syndrome coronavirus-1 (SARS-CoV-1), severe acute respiratory syndrome coronavirus-2 (SARS-CoV-2) or Middle East respiratory syndrome coronavirus (MERS-CoV).

12. The method of claim 10 or 11, wherein the estrogen receptor modulator is selected from the group consisting of: tamoxifen, toremifene, clomifene, or pharmaceutically acceptable salts and combinations thereof

13. The method of claim 12, wherein the estrogen receptor modulator comprises toremifene, or a pharmaceutically acceptable salt thereof

14. The method of any one of claims 10-13, wherein the anti-inflammatory agent comprises melatonin.

15. The method of any one of claims 10-14, wherein the estrogen receptor modulator and anti-inflammatory agent, or pharmaceutically acceptable salts thereof, are administered simultaneously or sequentially in any order.

16. The method of any one of claims 10-15, wherein the estrogen receptor modulator and anti-inflammatory agent, or pharmaceutically acceptable salts thereof, are provided in individual pharmaceutical compositions.

17. The method of any one of claims 1-16, wherein the estrogen receptor modulator and anti-inflammatory agent, or pharmaceutically acceptable salts thereof, are administered orally.

18. The method of any one of claims 1-17, wherein the estrogen receptor modulator, or a pharmaceutically acceptable salt thereof, is administered in a unit dosage formulations comprising 60 mg of the estrogen receptor modulator.

19. The method of claim 18, wherein the estrogen receptor modulator, or a pharmaceutically acceptable salt thereof, is administered once a day.

20. The method of any one of claims 1-19, wherein the anti-inflammatory agent, or a pharmaceutically acceptable salt thereof, is administered at a concentration of between 50 and 100 mg/day.

21. The method of any one of claims 1-20, wherein the anti-inflammatory agent, or a pharmaceutically acceptable salt thereof, is administered twice a day in unit dosage formulations.

22. The method of claim 21, wherein the unit dosage formulations comprise between 20 and 80 mg.

23. The method of any one of claims 1-22, wherein the subject is a human.

24. The method of any one of claims 1-23, wherein the subject has lung inflammation.

25. The method of any one of claims 1-24, wherein said subject is on a ventilator.

26. The method of any one of claims 1-25, wherein said subject has general body inflammation.

27. A composition comprising:
a selective estrogen receptor modulator, or a pharmaceutically acceptable salt thereof; and
an anti-inflammatory agent, or a pharmaceutically acceptable salt thereof.

28. The composition of claim 27, wherein the selective estrogen receptor modulator is selected from the group consisting of: tamoxifen, toremifene, clomifene, or pharmaceutically acceptable salts and combinations thereof.

29. The composition of claim 28, wherein the selective estrogen receptor modulator comprises toremifene, or a pharmaceutically acceptable salt thereof.

30. The composition of any one of claims 27-29, wherein the anti-inflammatory agent comprises melatonin.

31. The combination of any one of claims 27-30, wherein the selective estrogen receptor modulator, and a pharmaceutically acceptable salt thereof, and/or the anti-inflammatory agent, or a pharmaceutically acceptable salt thereof, are provided in a pharmaceutical composition.

32. A kit comprising:
a selective estrogen receptor modulator, or a pharmaceutically acceptable salt thereof; and
an anti-inflammatory agent, or a pharmaceutically acceptable salt thereof.

33. The kit of claim 32, wherein the selective estrogen receptor modulator is selected from the group consisting of: tamoxifen, toremifene, clomifene, or pharmaceutically acceptable salts and combinations thereof.

34. The kit of claim 33, wherein the selective estrogen receptor modulator comprises toremifene, or a pharmaceutically acceptable salt thereof.

35. The kit of any one of claims 32-34, wherein the anti-inflammatory agent comprises melatonin.

36. The kit of any one of claims 32-35, wherein the selective estrogen receptor modulator, or a pharmaceutically acceptable salt thereof, and/or the anti-inflammatory agent, or a pharmaceutically acceptable salt thereof, are provided in a pharmaceutical composition.

37. A method of treating a viral infection in a subject comprising:
administering a therapeutically effective amount of an anti-inflammatory agent, or a pharmaceutically acceptable salt thereof, to the subject,
wherein the subject is infected with a virus.

38. The method of claim 37, wherein the anti-inflammatory agent, or a pharmaceutically acceptable salt thereof, comprises melatonin.

39. The method of claim 37 or 38, wherein the estrogen receptor, or pharmaceutically acceptable salt thereof, is administered orally.

40. The method of any one of claims 37-39, wherein the anti-inflammatory agent, or a pharmaceutically acceptable salt thereof, is administered at a concentration of between 50 and 100 mg/day.

41. The method of any one of claims 37-40, wherein the anti-inflammatory agent, or a pharmaceutically acceptable salt thereof, is administered twice a day in unit dosage formulations.

42. The method of claim 41, wherein the unit dosage formulations comprise between 20 and 80 mg of the anti-inflammatory agent, or a pharmaceutically acceptable salt thereof

43. The method of claim 41 or 42, wherein the unit dosage formulations comprise a first unit dosage formulation comprising between 20 and 40 mg of the anti-inflammatory agent, or a pharmaceutically acceptable salt thereof and a second unit dosage formulation comprising between 40 and 60 mg.

44. The method of any one of claims 37-43, wherein the virus is a coronavirus.

45. The method of any one of claims 37-44, wherein the virus is selected from the group consisting of severe acute respiratory syndrome coronavirus-1 (SARS-CoV-1), severe acute respiratory syndrome coronavirus-2 (SARS-CoV-2), Middle East respiratory syndrome coronavirus (MERS-CoV), and Ebola virus.

46. The method of any one of claims 37-45, wherein the viral disease is human coronavirus disease 2019 (COVID-19).

47. The method of any one of claims 37-46, wherein the subject is a human.

48. The method of any one of claims 37-47, wherein the subject has lung inflammation.

49. The method of any one of claims 37-48, wherein said subject is on a ventilator.

50. The method of any one of claims 37-49, wherein said subject has general body inflammation.
